(19) United States
(12) Patent Application Publication
(10) Pub. No.: US 2008/0275295 A1
(43) Pub. Date: Nov. 6, 2008

(54) METHODS OF USING PERICARDIAL INSERTS

(76) Inventor: Michael Gertner, Menlo Park, CA (US)

Correspondence Address: SHAY GLENN LLP, 2755 CAMPUS DRIVE, SUITE 210, SAN MATEO, CA, 94403, US

(21) Appl. No.: 11/777,262

(22) Filed: Jul. 12, 2007

Related U.S. Application Data
(60) Provisional application No. 60/927,204, filed on May 1, 2007.
(60) Provisional application No. 60/916,262, filed on May 4, 2007.

(51) Int. Cl. A61F 2/02; A61B 17/32; A61M 29/02

(52) U.S. Cl. 600/37; 604/22; 604/96.01

(57) ABSTRACT

Devices, systems and methods are provided which are capable of applying pressure and constraint to the heart and use the pericardium to assist in the application of the pressure and force to the heart.

### CROSS REFERENCE TO RELATED APPLICATIONS

This application claims priority under 35 U.S.C. §119 to: U.S. application Ser. No. 60/927,204 of Michael Gertner, entitled “PERICARDIAL INSERTS AND METHODS OF USE” and filed on May 1, 2007; and U.S. application Ser. No. 60/916,262 of Michael Gertner, entitled “PERICARDIAL INSERTS AND METHODS OF USE” and filed on May 4, 2007, the disclosures of which are incorporated herein by reference.

### INCORPORATION BY REFERENCE

All publications and patent, applications mentioned in this specification are herein incorporated by reference to the same extent as if each individual publication or patent, application was specifically and individually indicated to be incorporated by reference.

### FIELD OF THE INVENTION

The present invention relates to methods and devices for treating heart failure.

### BACKGROUND OF THE INVENTION

Heart failure is a disease reaching epidemic proportions in the United States and the rest of the world. Over 5 million people in the US and 10 million people across the world suffer from heart failure. These numbers are increasing yearly due to improved technology to treat myocardial infarctions and coronary artery disease.

As the heart fells to function properly, it tends to expand over time to compensate for decreased ability to pump blood, leading to further heart failure and creation of a downward spiral ultimately leading to end stage heart failure and death or need for a heart transplant.

Proposed solutions to prevent cardiac dilation involve placement of meshes or nitinol sleeves (so called restraint devices) over the epicardium to prevent dilation and prevent the downward spiral. These devices are placed around the heart to apply a pressure on the heart wall. Because they are placed around the heart, an increase or decrease in tension on one region of the constraint device translates to an equal increase in tension on another region of the heart. Like a belt, an increase in the tension on one side causes an equal increase in tension on the other side of the belt. This is a major limitation of these devices because die right side cannot tolerate too high a pressure or it will be unable to fill. A further limitation of these devices is that they are not adjustable (reversible or titrateable) and are not removable from around the heart once they are placed because the materials that are used to produce these devices can induce tremendous scarring and inflammation. Furthermore, a major surgery is required to place them around the myocardium (sternotomy or thoracotomy).

Anatomically, the heart has four primary layers, the endocardium (blood contacting surface), the myocardium (muscle), the epicardium (the shell just outside the myocardium), and the pericardium (the outer covering of the heart). There exists a potential space between the pericardium and the epicardium (pericardial space) which can be filled with fluid. Sometimes, the fluid pressure is too high in the acute setting and the heart cannot expand. Typically, when the fluid pressure is uniform, the vena cava, the right atrium, and the right ventricle are the first structures which are compromised. These structures are compromised at pressures of about 10-20 mm Ha and a volume of less than 100 cc in the acute setting.

### SUMMARY OF THE INVENTION

The present invention includes devices and systems which are capable of applying pressure and constraint to the heart and use the pericardium to assist in the application of the pressure and force to the heart.

Aspects of the invention relate to a method of managing a heart failure patient. Anatomically, the patient has skin, costal cartilage, xiphoid and a heart. The heart has a left ventricle, a right ventricle, a left atrium, a right atrium, an epicardium, a pericardium and a pericardial space between the epicardium and the pericardium. The method of managing heart failure in the patient includes placing a support structure between the epicardium and the pericardium such that a force is transmitted from the pericardium through the support structure to a selected region of the epicardium, and leaving the support structure in place between the epicardium and the pericardium postoperatively.

The method step of placing the support structure may include placing a guide wire into the pericardial space through a puncture in the skin, positioning the guide wire over a region of interest of the heart, delivering the support structure over the guide wire to the pericardial space; and removing the guide wire. In some embodiments of this method involving placing a guide wire, the support structure includes a plurality of separate segments, and the plurality of separate segments is delivered over the guide wire one at a time. Some of these embodiments may further include interconnecting the separate segments after they are delivered over the guide wire. And in some of these embodiments, the separate segments are interconnected by joining-magnets located on the segments. In some embodiments, the segments are connected by elastic joints and are stretched longitudinally through a port, thereafter being delivered segment by segment out the distal end of the port.

The method step of placing the support structure may include placing a flexible sheath into the pericardial space through an opening in the skin, positioning the sheath adjacent to a region of the heart to support delivering the support structure through the sheath to the pericardial space, and removing the sheath. In some embodiments of this method step, the sheath is placed through an incision made in close proximity to the xiphoid. In other embodiments, the sheath is placed through the ribs; in other embodiments, the sheath is placed underneath the sternum from a small neck incision.

In some embodiments of the method of managing a heart failure patient, the support structure may further include an extrapericardial extension and wherein the extrapericardial extension further includes at least one securing portion that secures the support structure outside the pericardium.

In some embodiments of the method of managing a heart failure patient, the method may further include securing the support structure in place without sutures. In other embodiments of the method, the support structure is delivered through an opening in the pericardium no larger than about 1.5 cm. And in still other embodiments, the pericardium is maintained substantially intact while placing the support structure. In other embodiments, clips, sutures, locks, meshes, bolts, and/or fasteners are used to secure the device to the pericardium.

In some embodiments of the method of managing a heart failure patient, the support structure is expandable, and some of these embodiments, the support structure is expandable with a fluid. In some of these fluid-expandable support structures, the fluid expandable support structure is set at the time of implantation to reach a pressure of less than about 20 mm Hg when the heart is expanded during diastole. In some embodiments where the support structure is expandable, support structure is constructed to substantially cover one of the ventricles but not the other. In some embodiments, the fluid is water or saline; in some embodiments, the fluid is a gel; in some embodiments, the fluid is a gas; in some embodiments, the fluid is a curable gel.

In some embodiments of the method of managing a heart failure patient, the support structure applies a force substantially only to the left ventricle and not to the right ventricle.

In some embodiments of the method of managing a heart failure patient, the method further includes the step of adjusting said support structure such that said support structure transmits less than 30 mm Hg to the selected region of the epicardium through transfer of force from the pericardium through the support structure to the selected region of the epicardium. In some of these embodiments, the selected region of the epicardium is the left ventricle. In some embodiments, the selected region of the epicardium includes at least a portion of one of the atria,

Some embodiments of the method of managing a heart failure patient further include removing the support structure. Some embodiments further include adjusting the support structure to modulate a therapeutic effect of the support structure. And in some embodiments, the structure further includes an electrical conducting portion and said electrical conducting portion is activateable after implantation to create a desired therapeutic effect. In other embodiments, the support structure transmits other types of energy such as RF, ultrasound, light, heat, mechanical waves, suction, vibrations, and/or microwaves. In some embodiments, a subcutaneous port attached to the device through a connector in the pericardium stores energy and sends energy to the support structure. The energy can also be used to power an electronic circuit associated with the port, for pacing and sensing applications.

The invention further relates to a method of cardiac treatment that includes providing an implantable insert having an inflated state and a deflated state, placing the insert into a patient's body in the deflated state and positioning the insert over a selected region of the patient's heart, inflating the insert to put pressure on the selected region without applying substantial pressure to other regions of the heart; and maintaining pressure in the insert for an extended period of time to create a desired therapeutic effect on the region.

In some embodiments of the method of cardiac treatment, the desired therapeutic effect includes inhibiting cardiac dilation of the region.

In some embodiments of the method of cardiac treatment, the insert is placed between the epicardium and the pericardium.

Some embodiments of the method of cardiac treatment include deflating and removing the insert. In various embodiments of this method, the pressure is maintained in the insert for at least 1 month before removal. In the embodiments, pressure is maintained for at least 6 months before removal. And in still other embodiments, pressure is maintained for at least 2 years before removal. In some embodiments, the pressure within the insert is optimized, titrated, removed, and/or inserted over lime through percutaneous access of the port through the skin.

In some embodiments of the method of cardiac treatment, pressure is maintained in the insert such that it does not exceed a maximum of about 5 mm Hg, in others it does not exceed a maximum of about 25 mm Hg, and in still others, it does not exceed a maximum of about 40 mm Hg. In some embodiments of the method, a minimum pressure of about 5 mm Hg is maintained in the insert, in others a minimum pressure of about 10 mm Hg is maintained in the insert, in others a minimum pressure of about 20 mm Hg is maintained in the insert, and in still others a minimum pressure of about 5 mm Hg is maintained in the insert. In some embodiments, the pressure inside the insert is sensed using a pressure sensor associated with the support structure.

In some embodiments of the method of cardiac treatment, the selected region is the left ventricle.

The method of cardiac treatment may further include placing a reservoir port in the patient's skin and changing the pressure in the insert by adding fluid to or removing fluid from the insert through the port.

The method of cardiac treatment may further include placing a fluid pump in the patient's body in fluid communication with the insert to automatically pump fluid into the insert upon external command or through an internal feedback system.

The method of cardiac treatment may still further include placing at least one sensor in the patient's body for monitoring a parameter associated with the insert. In some embodiments the parameter is pressure, in others it's strain, in still others it's volume.

The invention further relates to a system for treating a heart; the system includes an inflatable support structure configured for implantation in a pericardial space, the support structure configured to transfer a force from the pericardium through the support structure to a selected region of the epicardium, the support structure further configured to be deliverable to the pericardial space through an opening in the skin no larger than about 1.5 cm; and an implantable tube in fluid communication with the support structure for providing fluid to inflate the support structure.

In some embodiments of the system for treating a heart, the support structure includes a plurality of compartments, each compartment being inflatable to a different pressure. In other embodiments, the system further includes a plurality of inflatable support structures. In some of these embodiments, the system further includes a plurality of implantable tubes, each of the tubes in fluid communication with one of the support members.

In some embodiments of the system for treating a heart, the support structure includes a pacing electrode configured to contact the epicardium.

Some embodiments of the system for treating a heart further include an implantable fluid pump in fluid communication with the implantable tube for delivering fluid to the support structure. Still others include an implantable sensor.

Some embodiments of the system for treating a heart further include an implantable system which inflates or deflates the support structure based on measureable parameters related to the support structure.

In some embodiments of the system for treating a heart, the support structure includes a composite material. In some of these embodiments, the composite material includes an elastomer and a second material to create a curvature to conform to the heart shape. In some embodiments, the composite material includes an elastomer and a coating. In some embodiments, the coating includes a hydrophilic coating, and in some embodiments the coating is a fibrosis-inducing coating. In other embodiments, the coating is conductive and is configured to transmit electrical energy to the epicardial surface. In some embodiments, the coating is different on different regions of the support structure. In other embodiments the composite material includes a shape memory alloy. In other embodiments, the support structure with a composite material includes a coating with a pharmaceutical molecule attached, and in still others, the support structure is configured to release pharmaceuticals.

The invention further relates to a heart-restraining device that includes an expandable support configured to deploy from an access sheath smaller than about 2 cm into a pericardial space around a heart, the support configured to expand into a heart-restraining configuration upon instillation of a fluid into the support, the support further configured to be implanted for an extended period of time to restrain the heart.

In some embodiments of the heart-restraining device, the expandable support is configured to encircle a portion of the epicardial surface of the heart. In other embodiments, the expandable support includes a composite material. In some of these embodiments, at least one portion of the composite material induces a shape change in the support. In other embodiments the composite material induces a desired biologic effect around said device.

In some embodiments of the heart-restraining device, at least one portion of the support is adapted to transmit energy to a portion of the heart or pericardium.

In some embodiments of the heart-restraining device, the support has a width and a thickness, and wherein the width is at least two-fold greater than the thickness, in others, the width is at least five-fold greater than the thickness, and in still others, the width is at least ten-fold greater than the thickness.

The invention still further relates to a heart restraining device that includes an expandable support configured to deploy from an access sheath smaller than about 2 cm into a pericardial space around a heart, the support configured to expand into a heart restraining configuration upon instillation of a fluid into the support, the support further configured to be implanted for an extended period of time to restrain the heart, and at least one radio-opaque marker permitting the device to be visualized from outside of a body.

Some embodiments of the heart restraining device include a plurality of radio-opaque or otherwise visualizeable markers located on, in or around the expandable support.

In some embodiments of the heart restraining device, the marker can be detected fluroscopically.

In some embodiments of the heart-restraining device, the support has a width and a thickness, wherein the width is at least two-fold greater than the thickness. In some of these embodiments, the width is at least five-fold greater than the thickness, and in others, the width is at least ten-fold greater than the thickness. In some embodiments, the support structure is produced from a membrane less than 200 microns thick. In some embodiments, the support structure is produced from a membrane less than 50 microns thick. In some embodiments, the support structure is produced from a material less than 25 microns in thickness. In some embodiments, the support structure is produced from a polyurethane, a silicone, PTFE, or combinations thereof.

In one embodiment, the device is implantable in the pericardial space through a sheath and through a small incision or puncture just under the xyphoid bone. In another embodiment, the device is implantable percutaneously through a small incision or puncture between the ribs.

In one embodiment, the device can be expanded with a fluid; after insertion into the pericardial space, the device in this embodiment is expanded to fill a selected space and volume in the pericardial space and also to apply a pre-determined pressure to the epicardium. As the heart expands and contracts, pressure builds inside the device, transmitting pressure from the pericardium to the myocardium and to the epicardial surface of the heart.

The device can be part of a system in which the device is adjustable through a reservoir port in the skin. Adjustment can be made either percutaneously with a needle to insert fluid into the port and then into the device, or through a transmitter which signals a mechanical pump to initiate pressure/volume adjustment of the device. The reservoir allows for titration of the volume/pressure inside the device and can also act as a module for sensing or other smart electronics related to the implanted devices.

One or more devices can be placed inside the pericardium. The one or more devices can be placed at different regions on the epicardial surface of the heart. The one or more devices can exert different independent forces on the epicardium through modifications of the material properties of the inserts or through differing volumes inside the devices. The one or more devices exert a force on the heart when the heart expands against the device and the device pushes on the inside surface of the pericardium.

The force(s) which are created by the devices are a combination of hydrostatic and material forces. That is, as the implant is compressed by the expanding heart (during diastole), the pressure inside the insert increases because of the tensile properties of the device. The hydrostatic pressure within the insert exerts a normal force on the epicardium. In addition, as the heart contracts during systole, the already expanded insert contracts down and exerts the stored potential energy on the myocardium.

In one embodiment, the device(s) augments the natural pericardial constraint applied by the pericardium, therefore acting as a composite material in combination with the pericardium to restrict expansion of the heart. In some embodiments, the device(s) are elastic, expanding during the diastolic cycle of the heart and contracting with the systolic cycle of the heart to exert a restrictive force during diastole and a corresponding compressive force during systole as the elastic potential energy leaves the device material.

The material used to manufacture the device is important. In some embodiments, the insert is produced from a hydrophilic material which absorbs greater than 10 percent water. In some embodiments, the hydrophilic material absorbs greater than 50 percent water and in some embodiments, the hydrophilic material absorbs greater than 90% water. In some embodiments, the insert material can absorb up to 99% water. By absorbing water, the material interlace with the epicardium is lubricious and advantageous in some embodiments. In some embodiments, the material is biodegradable. For example, in some embodiments, the material is biodegradable over about a 4 week period. In some embodiments, the material is biodegradable over about a three month period. In some embodiments, the material is biodegradable over about a six month period. In some embodiment, the material is biodegradable over about a one year period. In some embodiments, the material is biodegradable in a two year period or less. In some embodiments, the material is biodegradable upon photo-activation or another energy source.

In some embodiments, the device can be attached to one or more fill lines (e.g. tubes) operable to fill the one or more pericardial devices with a fluid. The fill line(s) can be permanently or temporarily implantable. The fill line(s) can be connected to one or more reservoirs to create a closed system with fluid inside both the insert and the reservoir. The reservoir(s) can be chronically implanted and/or fillable via puncture through the skin. Alternatively, the reservoirs can be automatically inflated or deflated with small pumps implanted under the skin in communication with the reservoirs. The pumps can be manually operated or automatically operated from within the patient or external to the patient.

In some embodiments, the devices can exert different surface forces on different regions of the heart by virtue of being composed of different materials or different material configurations; in some embodiments, the individual devices contain different amounts of fluid. In some embodiments, it is an object to control the pressure inside of the inserts. For example, it may be desirable to maintain the maximum pressure within the pericardial inserts to less than 25 mm Hg or in some cases to less than 15 mm Hg.

In some embodiments, a relief valve is provided on the fill lines so that a maximal pressure, if exceeded, triggers valve opening and release of hydrostatic pressure within the device.

The devices can be made from individual parts in some embodiments. For example, the inserts can be connected to one another by rigid or semi-rigid connectors which may or may not be fillable with fluid. The inserts can be connected by magnetic connectors in some cases. The magnetic connectors allow for self-assembly of the inserts inside the pericardial space.

In some embodiments, the device(s) can have integral sensors through which physiologic parameters are measured. For example, the sensors can measure the hydrostatic pressure inside the inserts or the hydrostatic pressure inside the pericardium. The sensors can measure the stress or strain on the inserts, on the surface of the heart, or on the inner surface of the pericardium. The sensors can detect electrical activity on one or more regions of the heart. The sensors can be placed on or in the devices, on the fill lines, on the reservoirs, or on any other structure attached to the inserts. Any or all of the sensors can communicate their data to a remote receiver or to one another.

When the physiologic sensor detects that the pressure is outside the desired range, the patient or physician can be alerted. In some embodiments, an automated pump is activated and fluid is added or removed from the device(s).

The inserts can be associated with electrodes. The electrodes can be integrally attached to the inserts or they can be attached directly to the epicardium or pericardium. The electrodes can communicate with the epicardium or other parts of the autonomic nervous system such as the parasympathetic or the sympathetic nervous systems. The electrodes can communicate with the sensors to create a communication or feedback circuit. Additional electronics, sensors, actuators, electrodes, and computer software can also be incorporated into the system. Radiofrequency transmitters can also be employed to relay information to the patient or to physicians involved with the care of the patient.

In some embodiments, the inserts are attached to the inner portion of the pericardium and in other embodiments, the inserts are attached to the epicardium or myocardium. Attachment can be achieved with sutures, with glues, or via tissue ingrowth into the electrodes. In some embodiments, energy, such as radiofrequency, microwave, or laser energy can be used to attach the devices to the pericardium or epicardium.

In some embodiments, the devices are elastic and expand as pressure is created within them.

In other embodiments, the device(s) are folded up for insertion into the pericardial sac through a sheath. In some embodiments, the devices are created such that they can be stacked longitudinally within a sheath and placed inside the pericardium one segment at a time.

In some embodiments, the device(s) are folded and placed in a sheath and then expanded when they are placed inside the pericardial space.

In some embodiments, the device(s) are linked to one another or conform to the shape of the heart such that when expanded the inserts create a restrictive force on the myocardium.

In some embodiments, the device(s) are used to treat one or more atria.

In some embodiments, the device(s) are used to treat one or more ventricles.

In some embodiments, the device(s) are used to treat atria and ventricles.

In some embodiments, one or more parameters of the inserts is measured over time (e.g. pressure or tension) and the volume of the insert is adjusted based on this recording. In some embodiments, the volume is adjusted by a physician and in some embodiments, the volume is adjusted automatically by an implanted pump. One example of an implanted pump is a piezo-electrically actuated pump.

In some embodiments, a fluid such as saline is used to fill the expandable device(s). In some embodiments, a gas such as carbon dioxide, nitrogen, xenon, or air is chosen. In some embodiments, a fluid such as a hydrogel is used. In some embodiments, a pharmaceutical compound is included in the fluid in the insert and is slowly released into the pericardial space.

In some embodiments, a method is described in which the pressure or volume of fluid inside the inserts is adjusted based on measured tension on the skin of the inserts.

In another embodiment, a hydrophilic material is used for the skin of the insert and the hydrophilic material can absorb some of the fluid within insert. Examples of a hydrophilic material is polyurethane. Another hydrophilic material is cellulose or bacterial cellulose. In another embodiment, a primary material such as a polyurethane or FTPE is used and a hydrogel coating such as a poly-ethylene glycol (PEG) placed on the primary material as a coating.

In some embodiments, a hydrophobic material is used for the skin of the insert.

In another embodiment, the insert has a porous or semi-porous material in which gas or materials can permeate. In some embodiments, the material is hydrophilic and gas permeable.

In another embodiment, a system is described in which the inserts comprise sensors and the information from the sensors is transmitted through the skin of a patient to a receiver outside the patient.

In some embodiments, the sensors cover the inserts or are placed inside the implants. In some embodiments, the sensors reside in the attached tubing or port or are hydraulically associated with the attached tubing or ports.

In some embodiments, the inserts are placed through a pericardial window or through a thoractomy or through a catheter placed into the heart via blood vessels.

In some embodiments, the inserts are coated with a material such as parylene, silicone, Dacron to alter the interaction between the surface of the insert and the epicardial surface.

In one embodiment, a method is described in which pressure in the insert is measured periodically from 1 day to 60 days depending on the patient and physician. In some embodiments, it may be desirable to adjust the pressure within the insert every 60-120 days. The desired pressure inside the pressure sensor may be less than 10 mm Hg or less than 20 mm Hg or less than 30 mm Hg. In some embodiments, a pressure between 10 and 20 mm Hg is desired and in some embodiments, a pressure between 0 and 10 mm Hg is desired. In this method of use, the pressure in the insert is obtained via internal or external pressure; the pressure in the insert is then adjusted by adding or removing fluid from the insert so as to correct the pressure toward the desired pressure.

In another embodiment, an internal pressure sensor complete with data logger is used to continuously monitor pressure within the insert and alert the patient or physician of pressures that are either too high or too low. In some embodiments, the physician or patient then adjusts the pressure in the insert based on the degree of deviation of the pressure from the desired pressure. In some embodiments, the internal pressure monitor communicates with an electrically active device either directly or indirectly. Examples of electrically active devices include drug delivery pumps, pacemakers, defibrillators, resynchronization devices, hydraulic pumps to pump fluid into or out of the inserts.

In another embodiment, a method is described in which a parameter associated with the pericardial insert is measured and the composite material properties of said pericardial insert parameter is adjusted based on said parameter associated with said pericardial insert. In some embodiments, the parameter is hydrostatic pressure. In some embodiments, the hydrostatic pressure is communicated wirelessly through a fluid port.

In another embodiment, a method of delivering a pericardial insert into a pericardial space is described in which said insert is delivered into the pericardium within a sheath, through the pericardial sac, and into the pericardial space. Said sheath is subsequently removed leaving the pericardial insert inside the pericardial space. In some embodiments, said sheath is <5 mm, in some embodiments, said sheath is <1.0 cm, and in some embodiments, said sheath is smaller than 2 mm. In some embodiments, said method further comprises closing a puncture in the pericardium created by the sheath. In some embodiments, said insert further comprises tubing which communicates with said insert and which is operable to fill said insert when fluid is introduced into said tubing. In some embodiments, said method further comprises a sealing ring which fits over said tubing and is operable to hold said tubing in a fixed position within the pericardium without a leak through the pericardium.

In some embodiments, said balloon comprises a first compliant material and a second more rigid and shaped material which defines a specific insert shape.

In some embodiments, the insert comprises integral magnets operable to bring two ends of the insert together; for example, two components of the insert can be placed into the pericardial space and then they can self-assemble via the magnets bringing two pieces of the insert together around the myocardium.

In some embodiments, the insert is filled with a medicament and the medicament (e.g. a nitrate or a beta blocker) is eluted into the pericardial space over time.

In some embodiments, the insert is coated with a material which improves the biocompatibility of the insert by prohibiting ingrowth or preventing effusion formation around the insert. In some embodiments, the insert is coated with a material which promotes ingrowth of fibrous tissue from the pericardium or from the epicardium.

In another embodiment, the insert is removable after a period of time >24 hours. In one embodiment, a material is chosen which resists ingrowth and capsule formation. In one embodiment, this material is a thin polyurethane. In one embodiment, the thickness of the thin polyurethane is less than 50 microns and is preferable less than 25 microns. In one embodiment, the insert material is silicone. In one embodiment, the insert material is a combination of silicone and polyurethane. In one embodiment, the insert can expand up to 100% in volume. In one embodiment, the insert can expand up to 200% in volume.

In one method of use, the insert is implanted for a period of up to 6 months while the myocardium heals after a myocardial infarction; in another method of use, the insert is placed in side the pericardial space for a period of up to 2 years to treat a chronic dilated heart.

In one embodiment, the insert encircles the myocardium and provides for circumferential support. In another embodiment, the insert rests against a portion of the myocardium to support a region of the heart.

In one embodiment, the pressure within the insert is maintained at a maximum of about 10 mm Hg. When 10mm Hg is exceeded, a receiver external to the patient is alerted. In some embodiments, this maximum pressure is about 20 mm Hg. In some embodiments, the maximum pressure is about 30 mm Hg. In some embodiments, the maximum pressure is about 40 mm Hg. In some embodiments, the maximum pressure is about 5 mm Hg.

In one embodiment, a system for controlling expansion of the heart comprises a nozzle operable to be inserted into the pericardium for introducing fluid into the pericardium in a controllable manner; a fluid line connected to the nozzle; a port coupled to the fluid line and adapted to be accessed so as to inject fluid through the fluid line and through the nozzle into the pericardium. In another embodiment, the system comprises a reservoir to store fluid. In another embodiment, the system comprises a sensor to sense pressure in the pericardium or sense another parameter related to the heart. In another embodiment, the port comprises a pressure sensor. In another embodiment, the system comprises an actuator to push fluid into the pericardium through the nozzle. In another embodiment, the system further comprises a fluid expandable structure. In another embodiment, the fluid expandable structure comprises a balloon.

In one embodiment, a method to treat heart failure is described in which fluid is placed into the pericardium or taken out of the pericardium through a port and fluid line. The pressure is sensed and the patient is assessed and fluid is again removed or placed into the pericardium to create a pressure.

In some embodiments, a system to treat a falling heart of a patient comprises at least a first structure adapted for placement into a pericardium of a patient wherein the structure is adapted to apply a first force to a first region of the heart and a second force to a second region of the heart, and wherein each force is adjustable from outside the pericardium. The first structure may contact the first region and not the second region. The first structure may be adapted to transfer force applied to said first structure from the pericardium directly to the epicardium of the heart. In some embodiments the system further comprises a second structure wherein the first structure contacts the first region and the second structure contacts the second region. The first structure may be adapted to transfer force from the pericardium to the first structure, the structure then transferring force to a structure outside of the pericardium. The structure outside the pericardium may be an expandable structure, and the structure outside of the pericardium may be expandable with fluid.

In some embodiments, a system to treat the heart of a patient comprises at least two distinct structures which are insertable into the pericardial space, and at least one tube which communicates with at least one of the structures, the tube being adapted to be placed outside the pericardium. At least one of the separate structures may be inflatable. The separate structures may be inflatable with independently implanted and independent tubes and/or may communicate with two independent access ports. In some embodiments, the two separate structures individually apply electrical current to the heart. One structure may contact the left ventricle while the other structure contacts the right ventricle. In some embodiments, the separate structures in combination substantially surround the heart but are not connected.

Another aspect of the invention provides a heart support, device including a material configured to deploy from an access sheath smaller than about 2 cm into a pericardial space, said support device being adapted to unravel and surround a portion of the heart upon deployment. The device may have a radius of curvature of between 2 and 10 cm. when unraveled. The material may be an expandable support made from a composite material. At least one portion of the composite material may induce a shape change in the support and/or induce a desired biologic effect around said device.

The device material may have at least one fluid fillable region, and the thickness of said material comprising said fluid-fillable material may be less than 100 microns, less than about 50 microns, or less than about 25 microns. In some embodiments, the width of the material is at least ten times its thickness. The material may be a polyurethane or a polyurethane blend. In some embodiments, the device may be sufficiently elastic so that the device can be stretched into an access sheath whose length is 2-3 times greater than the width of the device.

In some embodiments, the fluid fillable region may have dimensions of 15 cm or less by about 10 cm or less and when filled with less than 50 cc or less of fluid, does not have a thickness greater than about 1.0 cm when placed into the pericardial space. In some embodiments, the fillable region does not expand to a thickness greater than about 0.5 cm without an increase in pressure, and in some embodiments, the fluid fillable region undergoes an increase in pressure when the thickness of the device is decreased to less than 0.5 cm and said device (or one fluid region) is filled with fluid.

Another aspect of the invention provides a heart restraining system including an expandable support configured for the pericardial space, the support configured to expand into a heart restraining configuration upon instillation of a fluid into the support, the support further configured to be implanted for an extended period of time to restrain the heart, said support further being configured to expand in a substantially two-dimensional manner; and a tube (such as an endoscope) with at least one lumen, said tube adapted to received said expandable support. In some embodiments, the diameter of the tube is about 2.0 cm or less. In some embodiments, the expandable support is adapted to be placed in the tube so that it expands in a substantially horizontal manner, and in some embodiments the support is adapted to expand in a substantially two-dimensional manner. The tube further may also have a second lumen and/or a camera. The support material may be removable alter a period of about 60 days.

According to aspects of the invention, a method of remodeling a heart comprises forming a tissue capsule within an intrapericardial space of the heart and adjusting the pressure exerted by the the capsule on the heart. In some embodiments, the forming step composes placing a material into the intrapericardial space. The material may be an expandable device, such as an inflatable device. In some embodiments, the forming step comprises inflating the inflatable device. The adjusting step may comprise inflating or deflating the device. In some embodiments the placing step comprises delivering the material to the pericardial space through a delivery sheath having diameter of 2 cm or less. The placing step may further comprise moving the material from a delivery configuration within the sheath to a deployed configuration outside of the sheath and within the intrapericardial space. The material in its deployed configuration may have a greater length in at least one dimension than in its delivery configuration. In some embodiments, the method further comprises removing the material after forming the capsule. The adjusting step may comprise inflating or deflating the capsule. The method may further comprise reinforcing the capsule with a high viscosity or high molecular weight material.

In some embodiments of the invention, a method of remodeling a heart comprises inserting a delivery sheath info an intrapericardial space between a pericardium and a myocardium of the heart, the delivery sheath having a diameter of 2 cm or less. The method further comprises moving a pericardial device from a delivery configuration within the sheath to a deployed configuration within the intrapericardial space. The intrapericardial device in its deployed configuration has a greater length in at least one dimension than in its delivery configuration. The method further comprises controlling the pressure from the pericardium onto the intrapericardial device and from the intrapericardial device onto the myocardium. The method may further comprise permitting the intrapericardial device to move from the delivery configuration at least partially to the deployed configuration without outside actuation. In some embodiments, the method further comprises expanding the intrapericardial device from the delivery configuration to the deployed configuration. The expanding step may comprise inflating the intrapericardial device. In some embodiments, the intrapericardial device is loaded into the delivery configuration within the sheath, the loading step comprising lengthening at least 200% in another dimension from an at-rest configuration outside of the sheath. A tissue capsule may be formed around at least part of the intrapericardial device, and the size of the capsule may be adjusted. In some embodiments, the placing step further comprises visualizing placement of the device with a camera inside the pericardial space. The placing step may further comprise visualizing adhesions with a camera inside the pericardial space and cutting said adhesions from within the pericardial space.

In some embodiments, a system for remodeling a heart comprises a delivery sheath having a diameter of 2 cm or less. The system further comprises an intrapericardial device having a delivery configuration and a deployed configuration. The device in its delivery configuration is disposed within the delivery sheath. The intrapericardial device in its deployed configuration is adapted to be disposed outside the sheath and within a pericardial space of a heart. The intrapericardial device in its deployed configuration has a greater length in at least one dimension than in its delivery configuration. The system further comprises a delivery tool adapted to advance the intrapericardial device through the sheath and into a pericardial space. The intrapericardial device in its delivery configuration may have at least a 200% greater length in at least another dimension than in its deployed configuration. In some embodiments, the intrapericardial device comprises a shaping component adapted to control movement of the intrapericardial device from the delivery configuration to the deployed configuration. The device may comprise a first and a second region of material, the first region of material comprising the shaping component and being more rigid than the second region of material. The device may comprise an outer material surrounding a shape memory element. In some embodiments, the device comprises a sealed chamber containing a field and a fluid port communicating with the chamber. In these embodiments, the chamber contains more fluid in the device's deployed configuration than in the device's delivery configuration. In some embodiments, the intrapericardial device in its deployed configuration has a radius of curvature between 2.5 cm and 10 cm, and/or comprises a membrane having a thickness of 50 microns or less, or of 25 microns or less. The membrane may be formed at least in part from hydrothane.

In some embodiments, an intrapericardial device comprises a delivery configuration and a deployed configuration. The device in its delivery configuration is disposed within a delivery sheath having a diameter of 2 cm or less. In its deployed configuration, the device is adapted to be disposed outside a delivery sheath and within an intrapericardial space of a heart, and to have a greater length in at least one dimension than in its delivery configuration. The device comprises a shape memory material adapted to move the device at least partially from the delivery configuration to the deployed configuration. The device may he configured to have at least a 200% greater length in at least another dimension when in its delivery configuration relative to its deployed configuration. The device may comprise a shaping component adapted to control movement of the device from the delivery configuration to the deployed configuration. In some embodiments, the intrapericardial device comprises a first and a second region of material, the first region of material comprising the shaping component and being more rigid than the second region of material. The device may comprise an outer material surrounding a shape memory element. In some embodiments, the device comprises a sealed chamber containing a fluid and a fluid port communicating with the chamber. In these embodiments, the chamber contains more fluid in the device's deployed configuration than in the device's delivery configuration.

The intrapericardial device described above may have a radius of curvature between 2 cm and 6 cm in its deployed configuration. The device may comprise a membrane having a thickness of 50 microns or less, or 25 microns or less. The membrane may be formed at least in part from a hydrophilic elastomer. In some embodiments, the device comprises a delivery configuration and a deployed configuration. The device in its delivery configuration is disposed within a delivery sheath, and in its deployed configuration is adapted to be disposed outside the delivery sheath and within an intrapericardial space of a heart. In these embodiments, the device may further comprise a shape memory region and an inflatable region. The shape memory region may comprise a polymer material, a metal alloy and/or an elastomer. The shape memory region may be constructed so as to induce a shape which conforms to an epicardial region of the heart. The shape memory region may be constructed so as to assist expansion of the inflatable device after the device is deployed from the delivery sheath.

### BRIEF DESCRIPTION OF THE DRAWINGS

The novel features of the invention are set forth with particularity in the claims that follow. A better understanding of the features and advantages of the present invention, will be obtained by reference to the following detailed description that sets forth illustrative embodiments, in which the principles of the invention are utilized, and the accompanying drawings of which:

FIGS. 1A-C depict an insert with one or more compartments or a common compartment which can be connected via a valve. Reservoir communicates with the inserts via tubing and the inserts are fillable through the tubing and/or through the reservoir.

FIG. 1D is a schematic drawing showing the functioning of the inserts inside a pericardium.

FIG. 2 depicts an example in which there is no insert and the fluid reservoir communicates with the pericardial space and the pericardial space is filled with free fluid rather than an insert. The space remains fillable through the reservoir and the pressure can be maintained/controlled using the subcutaneously implanted reservoir.

FIG. 3 depicts a system in which the insert comprises electrodes or magnets or sensors within the inserts. The inserts are fillable through fluid lines and the sensors/magnets/electrodes are controllable or detected through the port reservoir connected to the inserts.

FIG. 4A depicts the insert outside of the pericardium. A coating or separate compartment is depicted on the insert. The coating can control the biologic reaction to the insert.

FIG. 4B depicts the insert outside of the pericardium in which there is an expandable component adapted for placement outside of the pericardium.

FIG. 5 depicts an insert which can self-assemble into a structure inside the pericardium.

FIGS. 6A-B depict methods for deploying an insert into position.

FIGS. 7A-C depict various embodiments of an insert being deployed.

FIG. 7D depicts embodiments where multiple smaller inserts are placed.

FIG. 8A depicts an insert positioned between the epicardium and the pericardium in a deflated state.

FIG. 8B depicts an insert positioned between the epicardium and the pericardium in an inflated state.

FIG. 8C depicts devices to stabilize the insert within the pericardial space.

FIGS. 9A-B depict a subcutaneous access port and an insert positioned to exert pressure only on the left ventricle.

FIGS. 10A-10B are various views depicting one insert embodiment.

FIGS. 10C-10P are various views depicting other insert embodiments.

FIGS. 11A-11F are various views depicting insert embodiments with composite materials and coatings.

FIG. 12 depicts an example in which there is no insert and the fluid reservoir/port communicates directly with a capsule created by a previously implanted insert within the pericardial space.

FIG. 13 depicts a method of using an insert with a composite material.

FIGS. 14A-E depict methods with which to use pericardial inserts.

FIGS. 15A-C depict various embodiments of inserts in combination with electrodes.

### DETAILED DESCRIPTION OF THE INVENTION

### Pericardial Insert

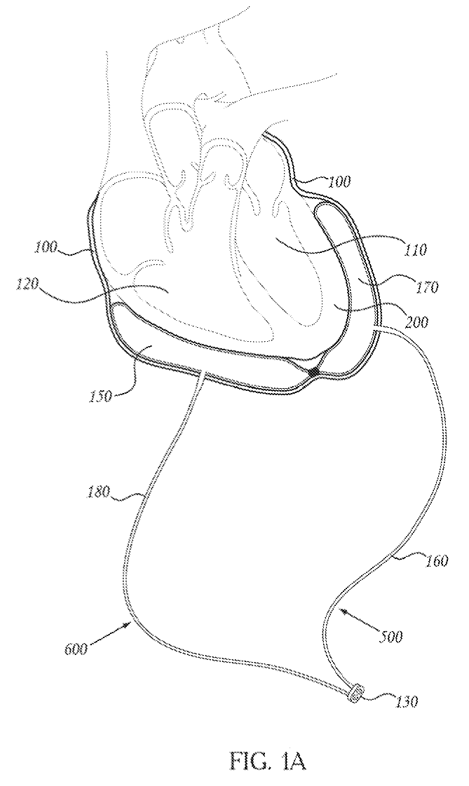

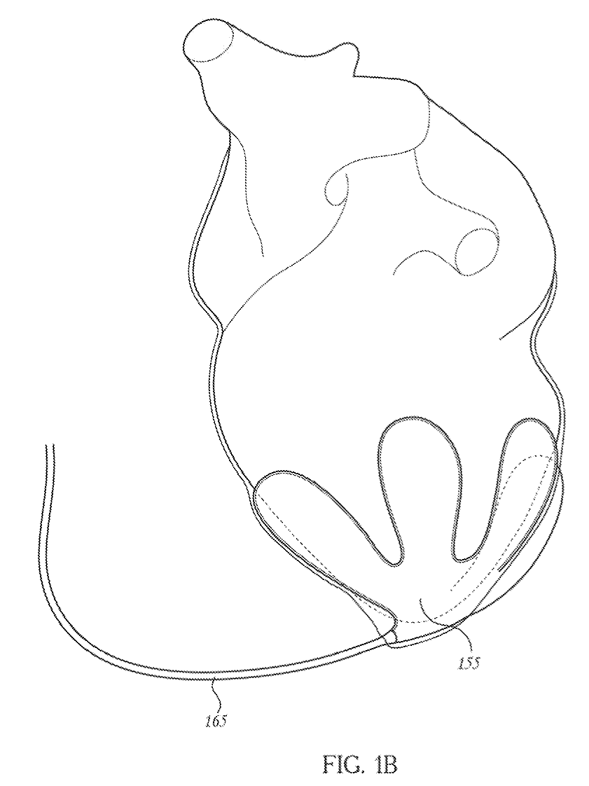

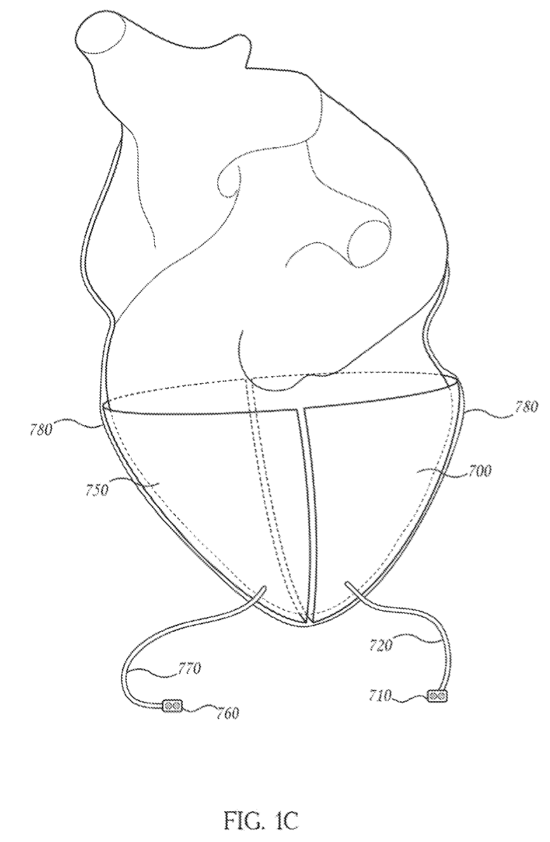

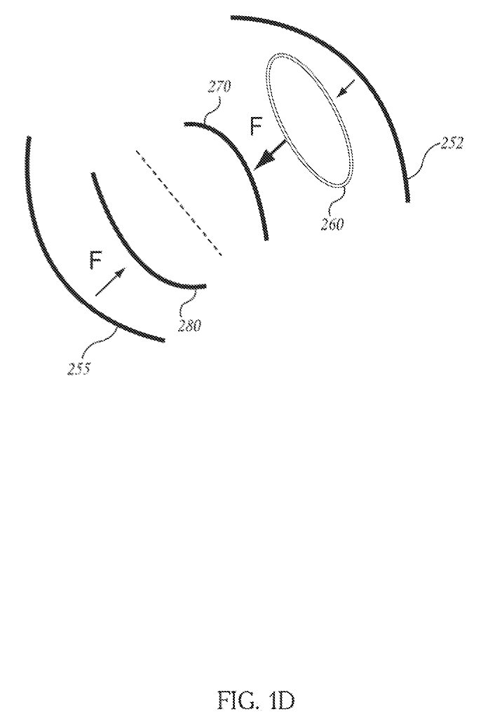

FIG. 1 depicts a heart 110, a pericardium 100, and an insert 150, 170 inside the pericardial space. The inserts 150, 170 or single insert (e.g. 150) are placed inside the pericardium to support a wall of the heart (e.g. the left ventricle) to prevent the wall from dilation, remodeling, or otherwise exhibiting maladaptive behavior; the insert may be placed as a chronically implanted insert (e.g. for 1-2 years), a sub-acute insert (e.g. 3 months in a dilated heart), or as an acute insert (e.g. immediately after a myocardial infarction and for 3-6 months thereafter).

In some embodiments, the insert can hold a fluid or gas within its walls; in some embodiments, the insert can be a solid material or a porous material. In some embodiments, the material is continuous but porous so that the natural fluid in the pericardial space flows in and out of the material to provide support to the epicardial surface. One or more inserts inside the pericardium can be filled with a fluid (liquid or gas) and the level of fluid or pressure inside the insert can be adjusted; if more than one insert is in place, then the fluid and/or pressure in each can be independently adjusted in the one or more inserts. The one or more inserts 150, 170 can exert a force on the heart when the heart expands against the insert.

In some embodiments, the insert is a chamber and the chamber contains separate devices such as stents or foams which may be dry but can exert a spring force on the wall of the heart. In some embodiments, the devices within the chamber are each filled with fluid and potentially different amounts of fluid so that different regions within the chamber can apply different pressures to the heart wall.

The pericardium 100 creates pressure on the insert when the heart expands; the pressure inside the insert in turn results in pressure on the myocardium 200. Pressure on the myocardium, as discussed above, can result in reversal or prevention of the maladaptive remodeling process. In the embodiment where inserts 150, 170 inside the pericardium are fillable with a fluid, the remodeling force can therefore be adjusted over time through fluid lines 160, 180 which are accessible through a port such as a subcutaneously implanted port 130. The material can be a thickened polyurethane or thickened silicone elastomer which supports the heart via force transduction through the wall. The material can be biodegradable or can be a hydrogel or may be both.

In some embodiments, inserts 150, 170 provide different forces to different regions of the epicardium. In one example, insert 170 is separated from insert 150 by a flow restrictor 190. Flow restrictor 190 can restrict the rate at which fluid may pass between inserts 150 and 170. In some embodiments, flow restrictor 190 may completely block fluid flow between inserts 150 and 170. In the above embodiments, compartments 150 and 170 are independent. For example, insert 170 can apply a greater or lesser force to the epicardium/myocardium than insert region 150. The forces applied to the heart can be different due to different hydrostatic pressures inside one of the insert regions or as a result of different materials used to produce the inserts. For example, the insert can be made from a thicker material on one side of the heart and a thinner material on the other side of the heart. When the heart expands during diastole, the side with the thicker material will result in a higher force applied to that region of the heart.

Pressures inside different regions of the insert or inserts can also be controlled independently through fill tubes 160, 180 which communicate with the insert regions 150, 170. Different forces can be applied to the different regions of the epicardium/myocardium depending on the amount of volume placed inside the insert(s) or inside different regions of the insert. For a given volume inside the insert, the myocardium will experience a given pressure. With a greater volume, the myocardium will experience greater pressure; for less volume, the myocardium will experience less continuous pressure. Such adjustability or titrateability is advantageous over time because the remodeling forces may need to be modified over time as the heart dilates or contracts.

In some embodiments of the invention, insert volumes may range from 10 cc to 90 cc or from 2 cc to 100 cc. In some embodiments, the desired volume is between 100 cc and 200 cc of fluid. A range of viscosities may be chosen for the fluid inside the insert. For example, water or saline solution may be the desired fluid inside the insert. Such fluids may have viscosities of around 0.75 to 1.25 cP (centipoise). In some embodiments, fluids such as dextran or other solutions containing a liquid and a larger molecule may be used. In some embodiments, the fluid is not homogenous and has a liquid phase and a solid phase, or a liquid phase and a gas phase. In some embodiments, if may be desirable to have a fluid with viscosity greater than 1.25 cP (for example, in the range from 1.25 to 100 cP). In some cases, thick fluids may be desired which have viscosities greater than 100 cP (for example, 101 to 1500 cP). For example, glycerol has a viscosity of 1490 cP. In some scenarios, it may be desirable to have a fluid with a viscosity less than 0.75 cP, for example down to 0.05 or 0.10 cP. In some scenarios, it may be desirable to use a gaseous fluid such as air, nitrogen, carbon dioxide, xenon, oxygen.

The inserts 150, 170 can be shaped or a material can be chosen so as to exert a force on a pre-determined area of the epicardium which of course transfers force to the myocardium in the form of shear stress and/or normal force; alternatively, the material can be chosen so that as the heart expands during diastole, the inserts are compressed and the compression pressure expands the material of the insert (dependent on the material properties of the insert); the increased pressure inside the insert is also transmitted to the epicardial surface and then to the myocardium. The pressure inside the insert can be used as a surrogate for the pressure applied to the epicardial surface. Additional parameters which can be used to estimate the force applied to the epicardium include the rate of pressure change within the insert, the maximum pressure change within the insert. The pericardium therefore acts to constrain the heart and the constraint is modified by the material properties of the insert. The material properties of the insert in turn may be modified by the filling status or filling material of the insert in the embodiment when the insert is fillable.

In another embodiment depicted in FIG. 1b, the insert has multiple fingers or extensions which surround different regions of the heart from inside the pericardium. For example, one finger can support the septum, another finger can support the left ventricle and a third finger can support the right ventricle. One or more of the fingers can communicate with one or more ports for independent control. In addition, one or more fingers may conduct current to the epicardium in embodiments where electrophysiology is combined with mechanical support.

In another embodiment depicted in FIG. 1C, inserts 700 and 750 are depicted as independent. In some embodiments, more than one insert is placed inside the pericardium and can be controlled independently of the others. In FIG. 1C, ports 710, 760 are shown which allow for independent instillation of fluid into the inserts 700 and 750. Access tubes 720 and 770 communicate with the inserts for instillation of fluid or medicines, electrical communication, pressure readings, etc. Any of the components of the system; for example, either the ports, access lines, or inserts can be compliant. In some embodiments, it is desirable for the extra-pericardial portions of the system to be more compliant than the intrapericardial inserts to shunt fluid away from the pericardium faster than would otherwise occur. In this embodiment, independent forces can be applied to different regions of the heart. For example, in FIG. 1C, insert regions 750 and 700 create forces which are different from one another on the epicardial surface. Each insert is controlled by a different access port 710 and 760. The inserts 750 and 700 may or may not contact each other and may or may not directly transfer force to one another. In some embodiments, more than two inserts are positioned in the pericardial space which may or may not contact one another. In a method to implant these one or more devices, the devices can be placed separately inside the pericardial space and allowed to heal independently of each other so that they attain their final position without connection and without concomitant placement.

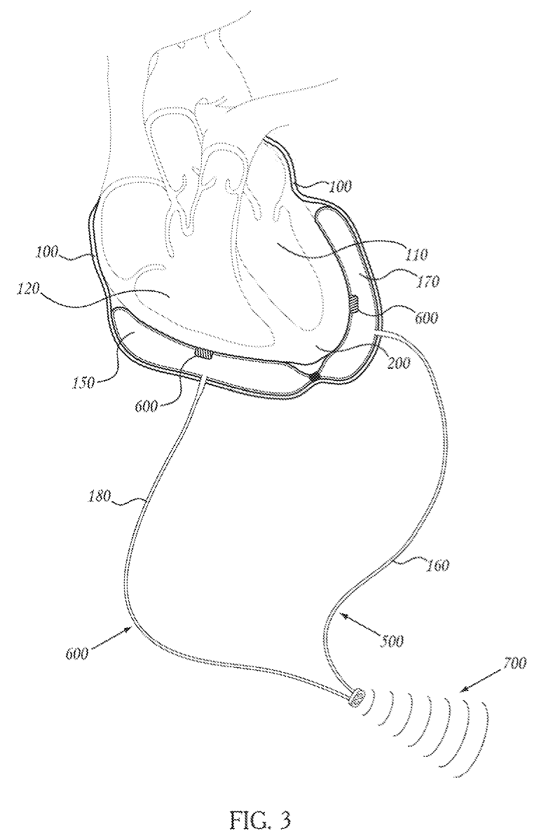

In FIG. 1D, a schematic of the device 260 is depicted in which force F is transduced to the wall of the myocardium of the left ventricle 270. The pericardium surrounding the left ventricle 252 pushes device 260 against the left ventricle 270. The force applied to the left side 270 is different than the force applied to right side 280, which is also transferred from the pericardium surrounding the right ventricle 255 to the right ventricle 230. This physiology is verified experimentally in Tables 1 and 2. The forces applied to the left side through the insert 260 are different from the forces applied to the right side 280. In some instances, an insert is placed on the right side (such as shown in FIG. 1C and FIG. 3) to further modulate the forces over time and independently of the left ventricle.

One or more independent inserts 150, 170 may be placed within the pericardium, each with its own compliance and material properties. For example, inserts 150, 170 may possess different material properties, sizes, or thicknesses so that the insert exerts less force on the right ventricle than the left ventricle or vice-versa (as an example). In some instances, inserts 150, 170 are placed close to a region of the left or right atria so as to decrease the amount of stress on the atria to treat and/or prevent arrhythmias by allowing the atria to decrease in size or to treat valvular disease. In some embodiments, the inserts are connected to one another within the pericardium by a connector 155 which links the inserts to one another. In some embodiments, the connector 155 acts as a fluid conduit between the pericardial inserts 150, 170. In some embodiments, a magnet is incorporated into the insert and acts as the connector by bringing components of the insert together inside the pericardium. Magnets placed inside of the inserts can also facilitate attachment of one or more inserts to one another. Magnets may be incorporated into the material of the inserts or may be secondarily attached with a glue to the inserts.

In some embodiments, one or more valves 155 are placed between the two inserts and the inserts are fluidly connected by the valve. The valve or valves can be opened or closed depending on the relative pressures within each of the inserts 150, 170. The valve or valves may be passively controlled based on pressure or may be actively controlled depending on the relative pressures inside the inserts. The valves may be controlled from a region external to the patient through a wireless transmitter. In some embodiments, the valve is a flow restrictor between the inserts, preventing or limiting the amount of flow between the inserts. The valves can create dynamic pressure profiles on the epicardium by different levels of flow restriction between them.

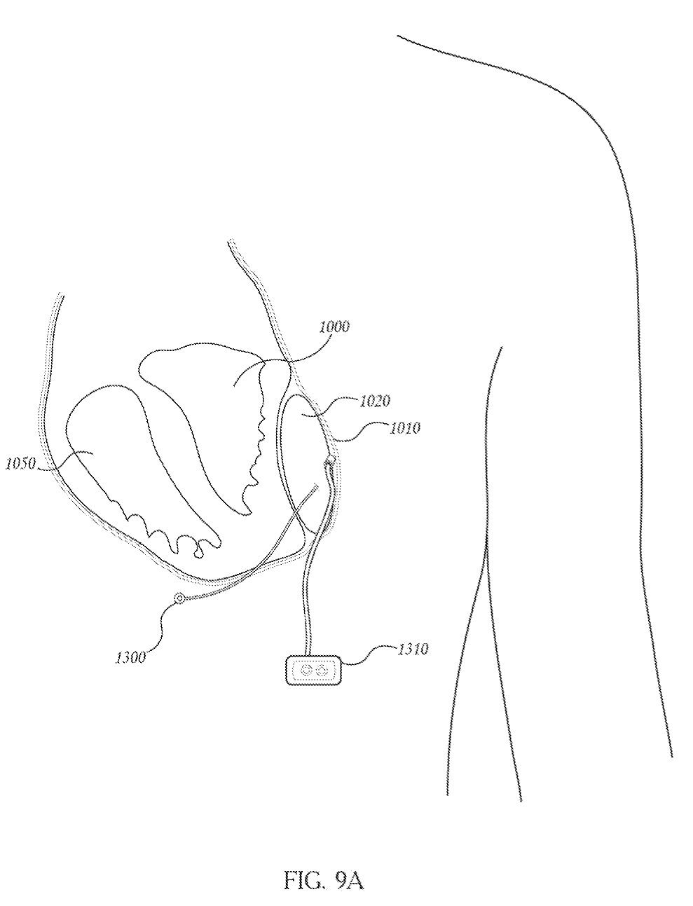

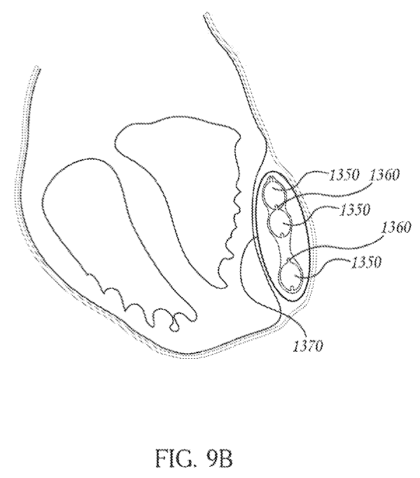

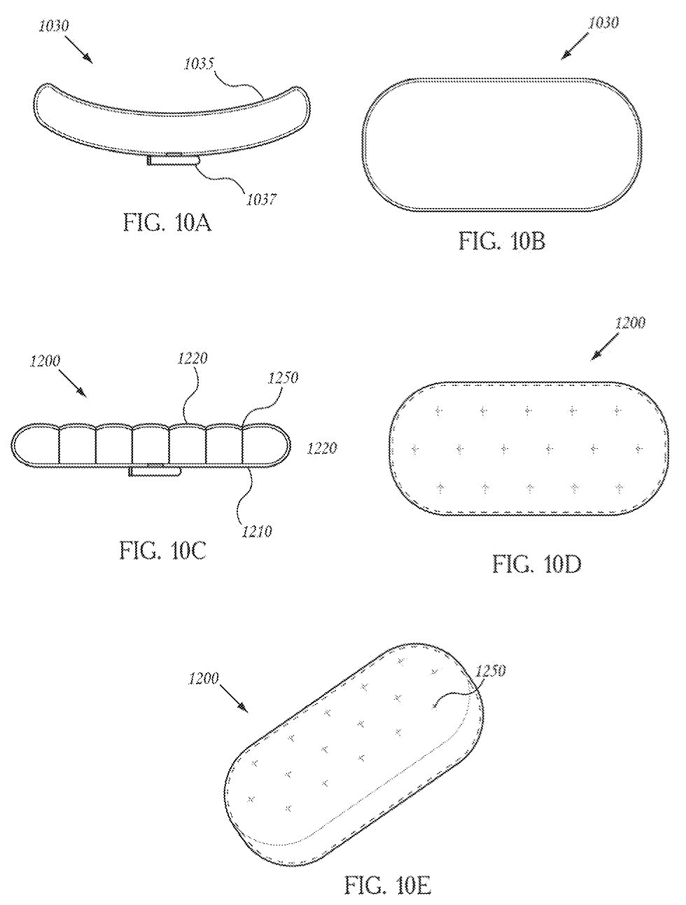

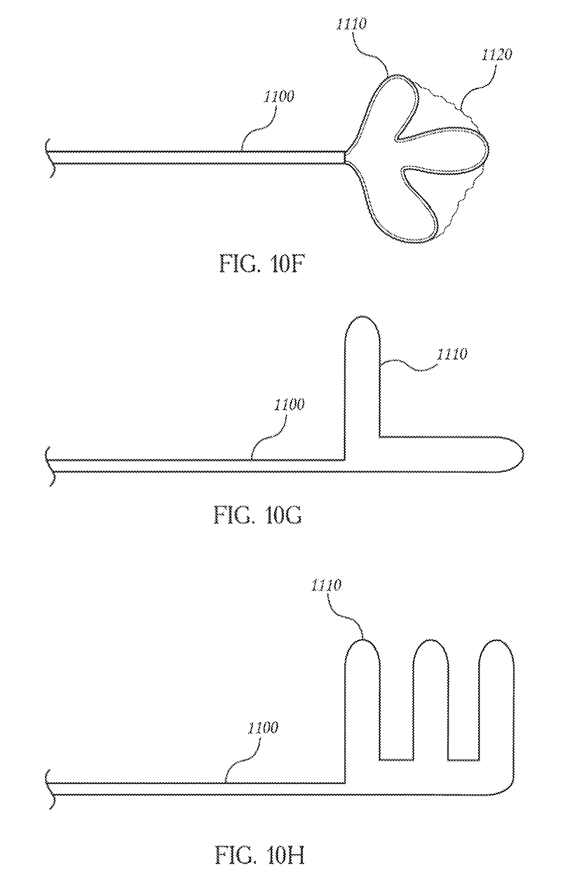

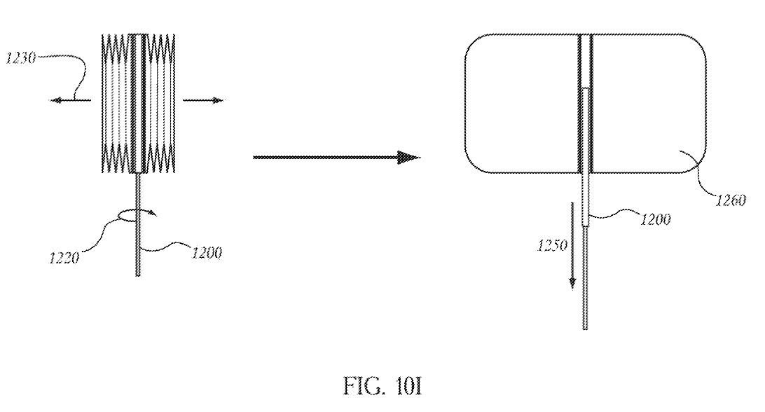

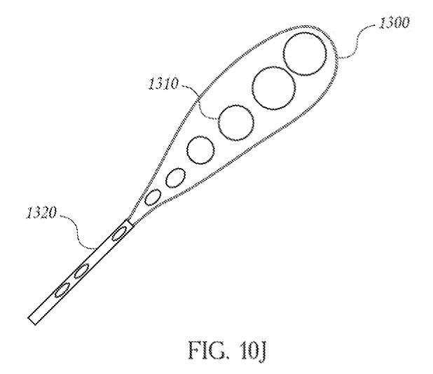

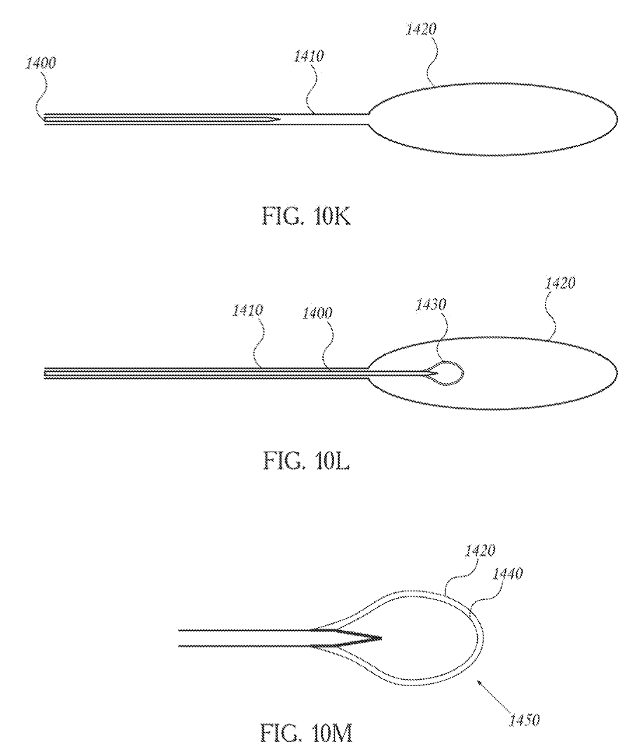

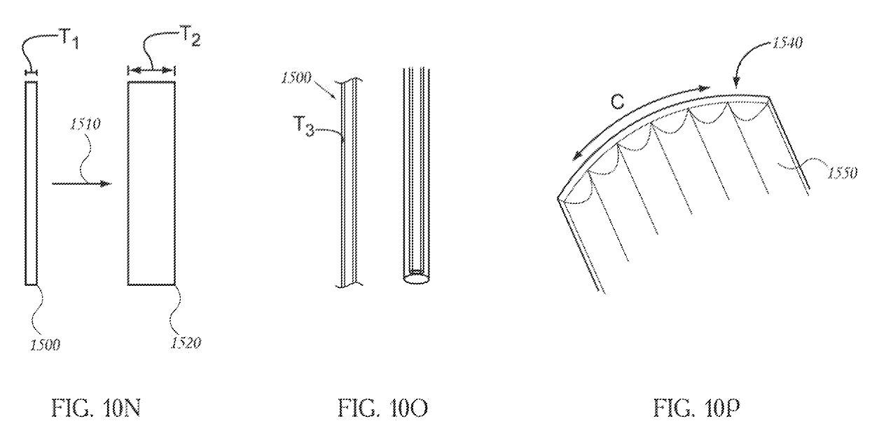

In some embodiments of this invention, only one insert is placed inside the pericardium. For example, an insert 1020 is placed between the left ventricle 1000 and the pericardium 1010, such as that shown in FIG. 9, and exerts pressure only on the left ventricle 1000. This allows the right ventricle to continue to expand freely against the pericardium 1010. The single insert 1020 can be shaped in a way to optimize force on the myocardium. For example, one shape is a C shape or a crescent shape which can grip the heart and apply a directional force, such as insert 1030 shown in FIGS. 10A and 10B. Another shape (not shown) is shaped like a baseball glove to hold the heart inside. Another shape (not shown) is a malleable shape in which the pericardium and myocardial forces shape the insert rather than the insert having a baseline shape. Another form is that of an air mattress 1200, such as shown in FIGS. 10C-10E. In this shape, the side 1210 facing the heart has a bubble contour 1250 which can more naturally fit the contour of the heart.

### Methods of Manufacture:

As an example, an insert was manufactured using a polyurethane blend (e.g. hydrothane 93A) from CardioTech International. An insert was created using this material by placing formed pellets (the way the material is sold by the manufacturer) into THF or DMAC (a solvent). A mold in the desired shape of the insert is then used to shape the implant; the mold is dipped into the hydrothane-solvent solution and dried to create the elastic insert which will be placed into the pericardial space. The neck of the insert may be defined by the mold or the insert may be manufactured with a wide mouth and then crimped over the fluid communication lines which communicate between the port and the insert.

Alternative materials include PTFE, silicone, or blends of these in combination with polyurethanes can be used.

In another manufacturing embodiment, the sheet of material is wrapped around a mold and the ends are heat sealed so as to create an enclosed volume. The balloon in this or any of the embodiments can be further modified such that the different regions are created by sealing different regions of the balloon so that it looks like an air-mattress when it is inflated.

The insert(s) 150, 170 may be ribbed or have many small bubbles along its surface. Such raised areas can ensure a relatively uniform distribution of pressure along the myocardium. One or more ribs can have greater or less thickness than the other ribs so that the compliance can be varied over the surface of the insert. In addition, the pressure within each rib or bubble can be adjusted independently over time. Ribs can be produced by heat sealing thicker pieces of material such as polyurethane to the inserts in strategic regions. Bubbles can be produced, by sealing (with direct heat or radiofrequency energy) the walls of the insert to itself in strategic regions. In some embodiments, the insert is heat sealed to itself in a substantially linear or feather-like pattern to create a curvilinear device upon expansion.

A coating can be applied to the surface of the insert after it is formed or before it is formed. For example, a hydrogel coating can be heat sintered to the surface of the insert or chemically attached to the surface of the insert. The coating can be applied to the inner surface of the insert or the outer surface of the insert. The coating can be applied prior to or after the formation of the insert. In some embodiments, a separate coating is applied to one region of the insert versus another region of the insert. In this embodiment, the insert can heal to the pericardium (for example) and remain fluid against the epicardial and myocardial surfaces.

The coating can applied by dipcoating methods or heat sealing methods or both. The coating can be a material surrounding the insert or a material partially applied to a region of the insert without surrounding the insert. In some embodiments, the coating is a second material. For example, the coating can be another polyurethane with a roughened surface and the second material can be heat bonded to the first material to create a composite material.

In another embodiment, the surface of the implant can be roughened after the insert, is manufactured. This rough surface can be produced by an etching process or by mechanical abrasion.

In another method of manufacture, the balloon shape is created inside of a blow mold. That is, the monomer is placed inside a mold and then a gas under pressure is pushed through the mold, expanding the polymerizing monomer. The material then takes the shape of the inside of the mold.

### Pressure Control of the Inserts

Pressure within the inserts can also be controlled by a valve external to the insert. In

FIG. 1, the valve or port 130 is implantable subcutaneously. In one embodiment, the valve is a reservoir with a membrane. The membrane can be a silicone membrane which is accessible through the skin with a needle. The needle punctures through the skin and then, through, the membrane; the silicone can self-seal after the needle is removed. The reservoir and membrane create a valve, the valve being accessed and “opened” when the needle passes through the membrane.

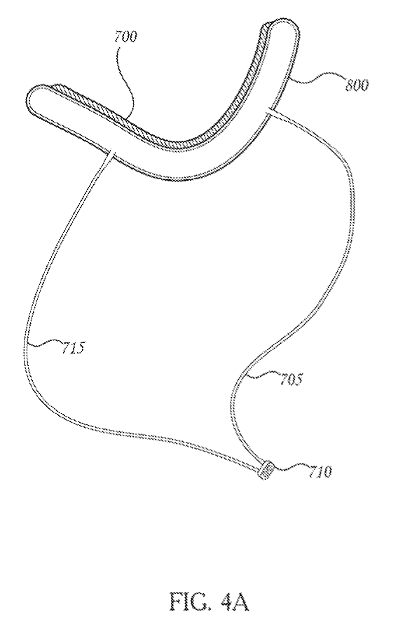

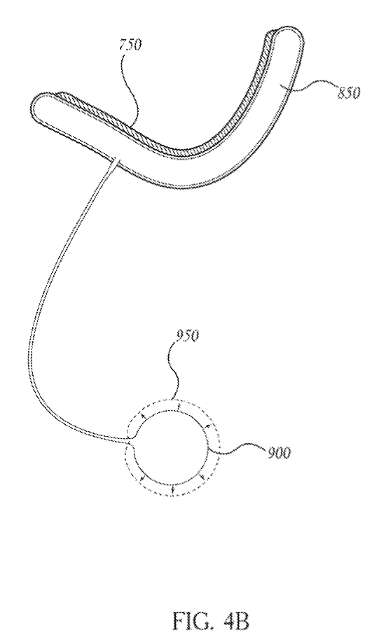

The port 130 can also contribute to the compliance of the overall system (FIG. 4B). For example, the port can be connected to a bladder or can itself be a compliant bladder and act as a low resistance reservoir for fluid leaving the insert inside the pericardium. Port 130 or an attached bladder can have multiple compartments which are filled with a fluid and increase or decrease the compliance of the system. These individual compartments can be controlled from a region external to a patient or can be controlled directly through the skin by the physician, with a hypodermic needle (for example).

FIG. 4b depicts a system with extxapericardial compliance built in. The insert 850 can be filled with fluid and the fluid can be shunted away from the heart during diastole vis-a-vis compliance built into the port 900. An expanded state 950 of the port is also depicted. In this embodiment, the device inside the pericardium can have various degrees of flexibility and the flexibility can be increased or decreased by having a low resistance reservoir outside of the pericardium. In this embodiment, the force from the pericardium is shunted to a region outside the pericardium rather than other parts of the pericardium or other structures surrounding the pericardium.

In these embodiments, the pressure or the force on the ventricle is maintained by the constant volume inside the insert-port system as well as by the materials included in the system. Some or all the materials of the system can be compliant, elastic, or rigid. The compliance of the system can also be modified by the viscosity of the material introduced into the system. For example, if a gas is introduced into the system, then the gas can be compressed upon expansion by the heart and would compress much more than a liquid would be compressed within the insert, therefore increasing the compliance of the system. The pressure or force can therefore be adjusted through adjustments in the volume of the system and the adjustments are performed by accessing the port and injecting fluid into the system or removing fluid from the system. In some embodiments, one or more valves may be required to control the pressure on different regions of the epicardial surface independently.

In further embodiments, the volume in the system is adjusted automatically through an implanted pump (as one example). The implanted pump communicates with the system and adjusts the volume in the system automatically. The implanted pump can also be manually controllable through the skin. For example, the pump can have a button on it which the physician can depress in order to change the resistance of a valve or increase the amount of fluid in the system.

### Inserts as Physiologic Sensors

In some embodiments, at least one sensor is provided which communicates with the insert or inserts. In this embodiment, the sensor is a pressure sensor, a strain sensor, a motion sensor, an accelerometer, a position sensor, a capacitance sensor, a resistive sensor, a temperature sensor, a pH sensor, an accelerometer, a pacing sensor, or any other type of sensor which detects a physiologic change on the insert or the heart. The sensors can be placed on the body of the insert, inside the insert, on the material which makes up the wall of the insert, or can be attached to another region of the heart away from the insert, creating a system of devices rather than one individual device.

Other examples of sensors are electrical sensors which sense currents or other electrophysiologic parameters. Sensors can be placed on the epicardium, the endocardium, or inside the myocardium to detect any of the physiologic or electrophysiologic parameters described above. Different sensors can be placed on or in different regions or portions of the heart. In some embodiments, sensors or electrodes are placed in certain regions of the heart and inserts are placed on other regions of the heart. For example, an insert can be placed on the left ventricle and a sensor placed or a therapeutic electrode placed on the right ventricle. In some embodiments, an insert and an electrode is placed on multiple regions of the heart.

The sensors can send signals through the patient to an external receiver or the sensor can send the signal to an infernal storage unit for download to an external unit at a later time. The internal storage unit can store and interpret the signals from the sensor. The internal storage unit can communicate with a receiver outside the patient or the internal storage unit can send data to an implanted software program which then can communicate with the automated fluid controlled system. Alternatively, the sensor can communicate with one or more pacing electrode systems on, in, or otherwise in communication with the heart.

Inserts 150, 170 can be connected to supply lines 160, 180, which allow for different amounts of fluid to be placed independently into one insert or the other. These lines can further be attached to a port 130 which enables injection of fluids into the inserts from outside the patient or to otherwise control functional parameters within the inserts. Port 130 enables physicians to adjust the pressure independently within each insert.

In another embodiment, the insert contains a pressure relief valve. For example, when the pressure inside the insert reaches a pre-determined pressure, fluid leaks out from the insert to decrease the pressure inside the insert.

### Self-Assembling and Buildable Inserts

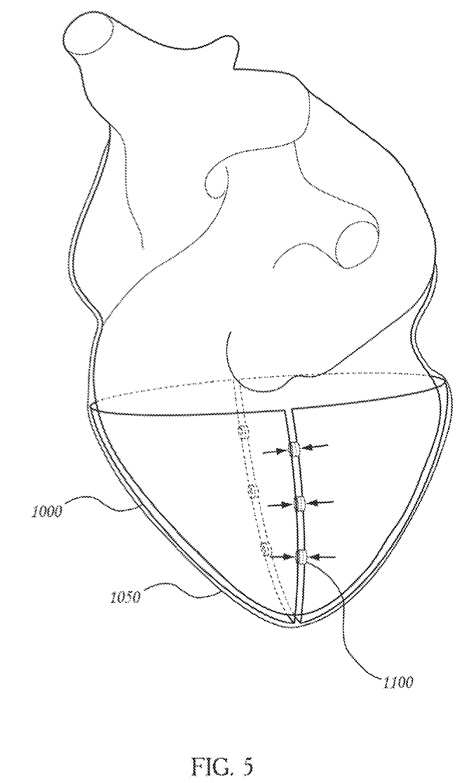

The inserts 150, 170 can include magnets as depicted in FIG. 5 (e.g. sumerium-cobalt or neodymium based alloy magnets). The magnets can be used to increase the force that the inserts apply to the epicardium and/or myocardium. The magnets inside the inserts can also be used to connect one or more inserts during implantation. For example, one or more inserts with magnets can be placed inside the pericardium and the inserts can then self-align within the pericardium when they are placed inside the pericardium so that they create a structure within the pericardium which applies a constraining force to the epicardium and/or the myocardium. The magnets may be placed anywhere inside or outside the inserts. Magnets may be placed anywhere inside the skin of the material. In one embodiment, small magnetic particles are placed inside the material insert or within the fluid inside the insert. In another embodiment, the magnets are placed along the edge of the insert so that the inserts can be held together like pieces of a puzzle.

FIG. 5 depicts a self-assembling insert in which magnets 1100 are placed on the edge of the insert structure 1000. The complex forms a structure 1000 inside of the pericardium 1050 through attraction (F) of the magnets and structural components inside the pericardial space. The magnets can be produced from a magnetic material which is biodegradable and/or biocompatible such as iron oxide. After implantation of the insert, these biodegradable magnets dissolve so that there will be no magnetic structures left inside the pericardium which would cause a problem during imaging studies such as magnetic resonance imaging. Biodegradable magnets can be used because the instability of the implant inside the pericardium is temporary until healing of the device occurs, after which the magnets are not necessary because the device is in place within the pericardial space. Alternatively, magnets are used for implantation purposes only and are removed after the immediate implantation.

In another embodiment, the magnets 1100 are strong enough to create the desired force F to allow the device to heal in place and to create restraint on the heart but they are small enough that there is no heating effect from an MRI scan. This window can be achieved because force F is relatively small particularly after healing occurs.

In one method of implantation, a first contracted, or undeployed portion of the final insert is placed inside the pericardial space, and then a second contracted, or undeployed portion of the final insert, is placed inside the pericardial space, thereafter allowing the individual portions to align or polymerize with one another to form a structure inside the pericardial space. The structure can encircle the heart or can create a force against one region of the heart. In some embodiments, more than two components of the implant come together to form the insert inside the pericardial space.

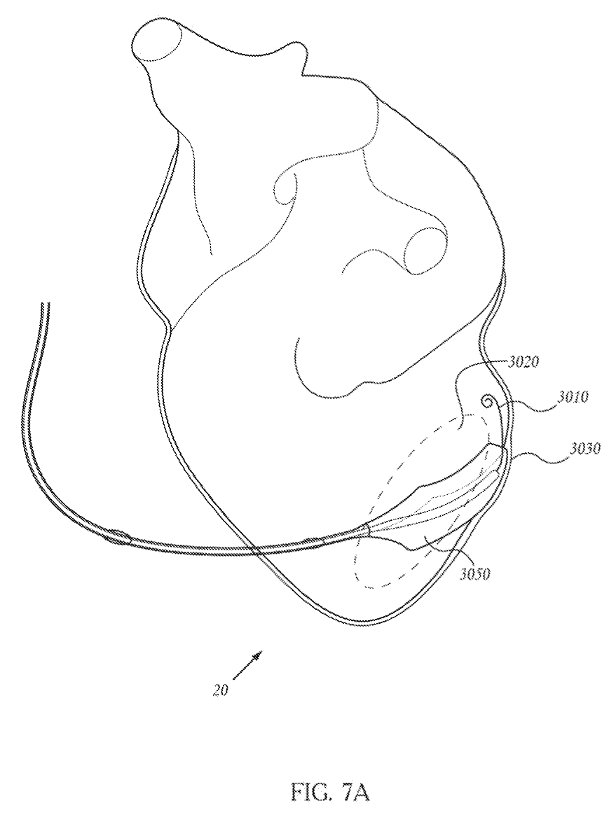

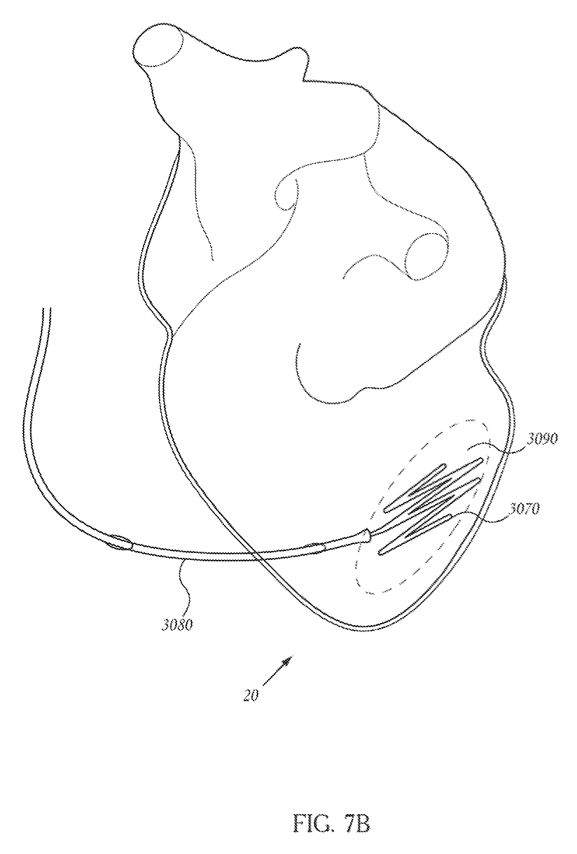

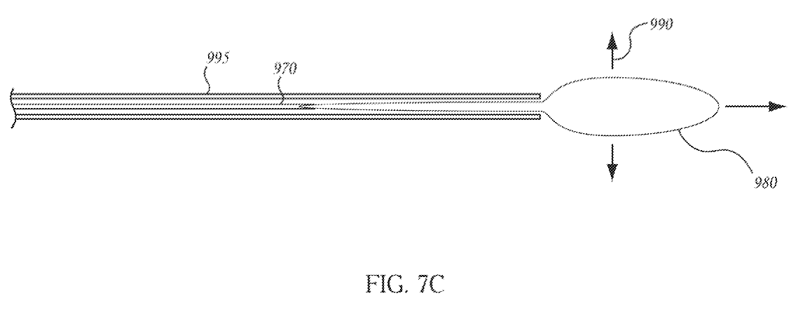

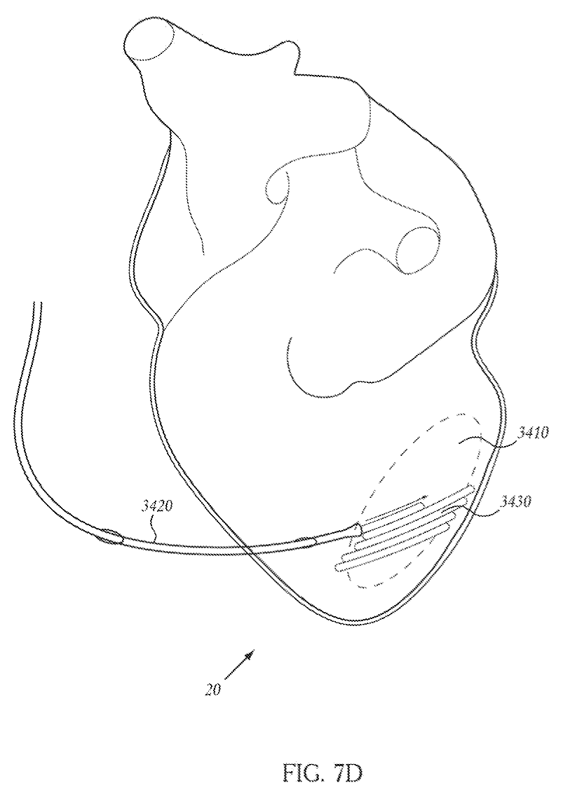

In other embodiments, the inserts do not quite self-assemble but are placed through an access tube 3420 piece by piece (FIG. 7D) and are built up within the pericardial space. In one embodiment, each component 3430 can be connected to each other one by one by an interlocking portion (FIG. 9B, 1360) to build an insert or support structure 1370 inside the pericardium. In another example, the individual pieces are attached to one another by material linking structures between them. Each individual piece can then be placed through the access tube and placed into the pericardium. If the material linking the structures is an elastic material, then the linking structures can be stretched as they are placed through the access tube like a rubber band. The elastic material will return to its pre-stretched configuration after the one or more pieces are inside the pericardium FIG. 7C; they will therefore be linked inside the pericardium.

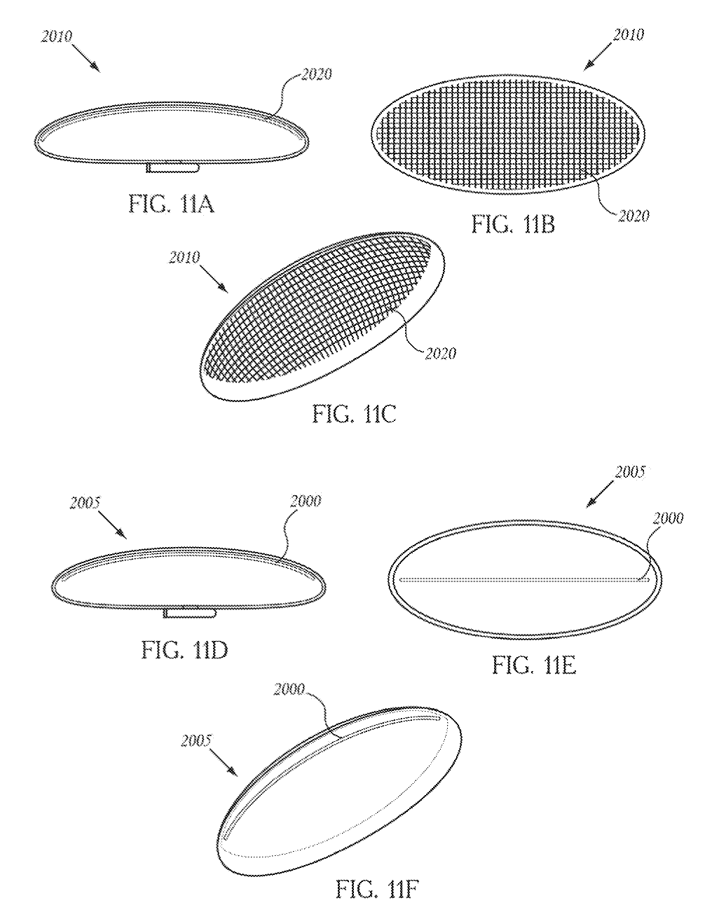

In a similar embodiment, several small inserts are placed inside the pericardial space side by side but not necessarily attached (FIG. 11F) to one another. In this embodiment, each insert can form a capsule around it after healing inside the pericardium; the summation of the capsules can create a fluid fillable chamber in which specific regions of the insert can be filled; therefore, some regions of the insert derived capsule can be modulated and other regions not modulated. The volume within each insert can be controlled independently such that the pressure along the epicardium can be modified locally even along the insert surface.

### Method Of Adjustment And Use Over Time:

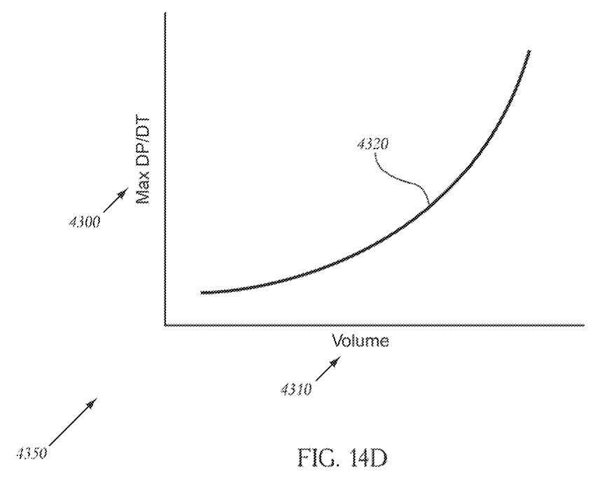

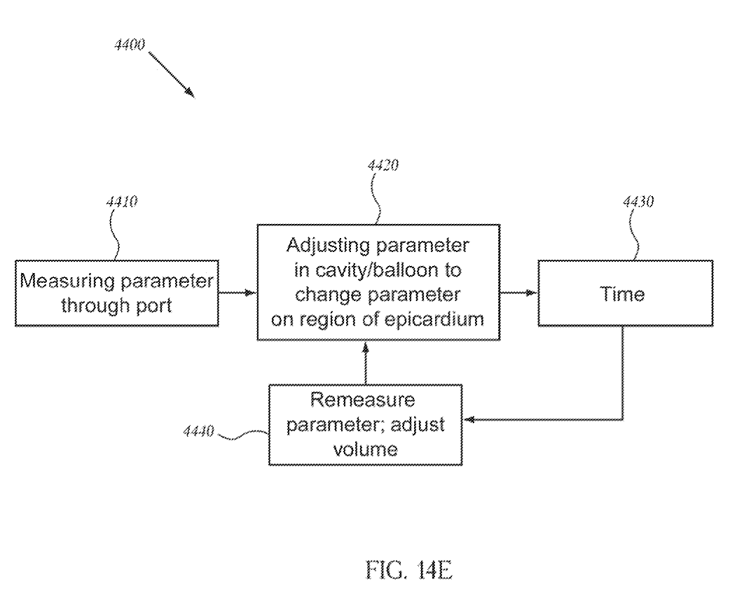

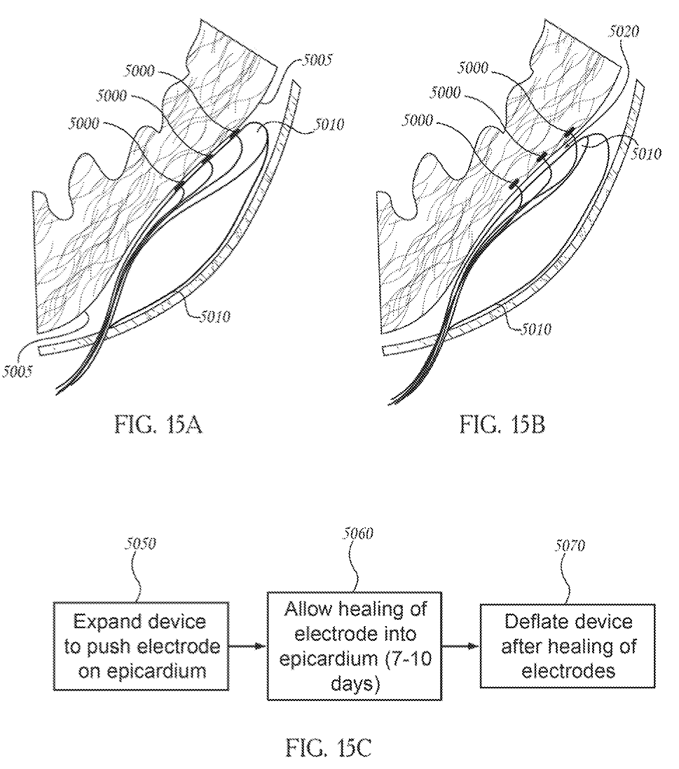

In one method 4400 (FIG. 14E), the force exerted by the inserts on the epicardium and myocardium of the heart is adjusted over time. The adjustment is performed in response to changes induced on the heart by the device and measured parameters 4410 related to the insert. For example, as the heart remodels and its diameter decreases over time 4430, the force on the myocardium will decrease as there may be more space in between the epicardium and the pericardium; increase in space translates to an increase in volume 4440 and a decrease in pressure on the epicardium. Similarly, as the pericardium remodels due to forces exerted on it by the insert, the volume between the epicardium and the pericardium decreases over time. Therefore, in one embodiment of this invention, volume and/or pressure within the insert is adjusted 4420 by injecting fluid or removing fluid from the insert. In one example, a subcutaneous port is used to perform these adjustments.

In another embodiment (FIG. 14D), the rate of pressure change inside the insert is used to adjust the force applied by the insert on the heart. For example, a sensor with a response time of at least 5 Hz is used to rapidly detect the pressure inside the insert. The rapid response of the sensor allows for the instantaneous pressure to be measured within the insert. From the instantaneous pressure, the rate of pressure change over the cardiac cycle can be accurately determined. The maximum rate of pressure change 4300 and the ability to rapidly detected pressure Is very important as opposed to a pressure averaged over a time greater than about ½ second or 2 Hz in which there could be inaccuracies due to rapid changes and shifts during the cardiac cycle. Depending on the material used for the insert, the pressure inside the insert may spike higher before it reaches its average pressure. Such dynamic changes can be determined using a fast cycling pressure sensor and plotting the maximum rate of pressure rise over time. In addition, dynamic pressure changes can represent the change in kinetic energy inside the insert over time. The rapid change from one pressure to another can create different pressure gradients related to momentum transfer which are likely important in the assessment of the force being applied to the heart from the insert and not reflected in a pressure reading. The graph 4350 in FIG. 14D depicts the maximum pressure change as a function of volume within the insert. The maximum pressure change increases with increasing volume within the insert. In some embodiments, it is desired to maintain the pressure change with time less than 20 mmHg/sec, in other embodiments, it is desired to maintain the pressure change over time to less than 40 mmHg/sec. In other embodiments, it is desired to maintain the pressure change over time to less than 10 mm Hg/second. Pressure change over time also correlates with force applied to the epicardium at a given instant. DP/DT correlates with momentum change and a force vector on the epicardium. DP/DT can correlate with a reverse remodeling effect in itself.

In another embodiment, the pressure is adjusted from a place external to the patient without accessing the device directly. For example, a valve can be activated wherein the valve permits transfer of fluid from one compartment of the insert to another compartment of the insert.

To facilitate adjustment, knowledge of the physiologic force or pressure or other parameter related to the inserts would assist the physician in making decisions. In one embodiment, this information is relayed to the patient or the physician so that decisions can be made based on the information.

In one embodiment, pressure inside the insert is measured over time to quantify the force being applied to the myocardium, in one embodiment, a pressure sensor is placed inside the insert. In another embodiment, a strain gauge is placed inside or outside the insert. These sensors can communicate with the subcutaneous port and then to the patient or physician. Alternatively, the sensors communicate directly with the patient or physician without going through the port. For example, the sensors are placed inside the insert or inside the skin of the insert. In one embodiment, the insert has strain gauges placed on or within the material of the insert. These inserts can be wireless transmitters which are activated by RF energy transmitted through the patient.

In another example, an imaging device is used to monitor the device over time; for example, echocardiography can be used in which a visualizeable fluid can be visualized within the insert.

In another embodiment, a method of use (FIG. 148) Is described in which a pressure-volume curve (FIG. 14B) of an inflatable insert is obtained at the time of implantation. 4020. A pressure-volume curve (FIG. 14C) is determined by placing a known volume inside the insert and then measuring the pressure inside the insert with a pressure monitor temporarily attached to the implantable port vis-à-vis a hypodermic needle through the skin, the hypodermic needle then connected to a pressure monitor; in some embodiments, other parameters associated with movement or force transfer inside the insert are utilized. As described above, a pressure monitor with a very rapid acquisition rate may be required in order to capture the proper transients within the insert.

An exemplary curve 4200 in FIG. 14C is shown which shows the pressure-volume curve at the time of implantation of the insert. Curve 4100 is the Pressure-Volume curve after some degree of healing 4030 has occurred. Exemplary data is shown in Table 2. Such time for healing may be 24 hours or 72 hours, or 5 days, or 10 days, or 30 days. After implantation, the insert has healed into place and the physician then re-measures 4040 the pressure-volume curve 4040 of the device with a similar methodology as performed at the time of implantation. The physician then readjusts the volume/pressure inside the insert so that the pressure is less than 15 mm Hg 4050. Experimental evidence shown in Table 2 indicates that the native compliance of the insert in combination with the healing around the insert effectively creates a composite system and that the pressure within the insert increases with time after implantation due to healing. The physician then must readjust the pressure by removing some of the volume within the insert. Although pressure is mentioned in this embodiment, any of the physiologic parameters disclosed in this application can be used to adjust the volume inside the insert. It is also possible that the insert acts as a tissue expander, effectively applying pressure to the healed capsule surrounding it and stretching the capsule over time.

In some embodiments where multiple compartments are included in the insert, individual heart regions can be affected differently than other regions. For example, pressure in the left ventricle can be affected differently than pressure on the right ventricle. Alternatively, pressure on the right ventricle dictates pressure and the left ventricle pressure is based off of the pressure needed for the right ventricle. In some embodiments, a different type of insert is placed in contact with the right ventricle as opposed to the left ventricle. In some embodiments, one of the atria is affected by the insert at one pressure and the left ventricle is also affected but the pressure is different than that applied to the atria.

In some embodiments, the pressure applied to the left ventricle is measured as less than 10 mm Hg inside the insert contacting the left ventricle and less than about 5 mmHg to the right ventricle as measured by the pressure inside the portion of the insert contacting the right ventricle. In other embodiments, pressure applied to the left ventricle by the insert.

### Method of Capsule Formation and Insert Removal Over Time

In another embodiment, a method 4300 (FIG. 14A) is described in which the insert (device) is removed or degraded over time. In this embodiment, the device is placed inside the pericardial space and sufficient time is allowed for healing inside the space. Healing 4320 allows for a capsule to form around the insert. The device is then removed 4330 after a period of time or the device is allowed to degrade 4330 over this period of time. The fill tube to the insert in some embodiments is not removed or degraded and therefore remains in place within a cavity created by the insert; the fill tube can then be used to inflate or deflate the capsule created by the insert 4340. Pressure or any other physiologic parameter can be measured inside the cavity and this pressure can be utilized to determine the amount of volume placed into the cavity over the therapeutic time. Certain materials can be added to the cavity to enforce the wall; for example, a cross-linking agent can be used to reinforce the capsule wall. The cross-linking agent can be applied selectively to the outer region of the insert versus the inner region of the insert. The cross-linking can be accomplished by radiofrequency generation to induce scar formation. The cross-linking can be accomplished by otherwise heating the region around the insert. In other embodiments, particular types of fluid can be used which will be retained within the capsule. Such fluids have higher viscosities or higher molecular weight materials in solution; exemplary solutions include dextran or albumin solutions.

In this embodiment, the capsule can be maintained by the access port and tube. For example, particular solutions or fluids can be introduced through the access port and into the capsule. The fluids can improve the integrity of the capsule or can coat the inside of the capsule. The fluids can polymerize inside the capsule or can cross link the wall of the capsule.

### Insert Shape

Inserts 150, 170 can be produced in various shapes including crescent shaped, banana shaped, curvilinear, ear-muff shaped, dumbbell shaped, or ring shaped. The inserts may be flat or may be curved with the surface of the heart or pericardium. In some embodiments, the shape of the insert is created using composite materials in which one material which acts as a spine or support structure to create a shape in combination with a second material attached to the spine.

The spine can be made from all standard materials including metals, plastic tubes, polymer meshes, etc.

In one embodiment the insert can have one or more fingers 1110 (FIG. 10H, 1B) which are expandable after the insert leaves an access sheath. The fingers can emanate from a central point like spokes from a hub (see FIG. 1B). In another embodiment, the insert is like a hand with fingers emanating from a palm shaped structure in the center.

### Insert Material

The inserts can be made from materials such as PET, PTFE, polyurethane, silicones, or combinations of these materials. In one preferred embodiment, the insert or inserts is made from a highly hydrophilic material such as polyurethane. The inserts can further be coated with hydrophilic coatings so that the insert slides within the pericardium. Another example of a material is a combination material of silicone and polyurethane. Such a composite material allows for the improved elasticity of silicone with the biocompatibility and strength of the polyurethane material.

In some embodiments (FIG. 10), the device is shaped like a square, in other embodiments the device is shape like a starfish, in other embodiments, the device is shaped like a circle.

In some embodiments (FIG. 10N) where the device is inflatable and when the device is filled with 100 cc of fluid or less, the thickness (T1, T2) is approximately 1 cm. In some embodiments, the thickness of the device when filled with 30 cc or less is approximately 1 cm. In some embodiments, the thickness of the device when filled with 30 cc or less of fluid is approximately 0.5 cm at atmospheric pressure (760 mm Hg). When the pressure inside the inserts is increased to 900 mm Hg, the thickness T1, T2 increases by less than 10%, the increase in volume being translated to the other dimensions of the insert. This restriction is an important design component in one embodiment because it ensures limited outward pressure on the pericardium. The thickness of the material (that is, the thickness T3 of the material which makes up the walls of the inserts) in these embodiments (10N and 10O) is preferably less than about 100 microns and in some embodiment, less than about 60 microns. The dimensions of the insert in these embodiments is less than about 15 cm in maximal diameter (width) and less than about 10 cm in height. In one example, if the diameter and height are 150 cm² and the thickness is 1 cm, the volume of the insert under atmospheric pressure is about 150 cm³. If the pressure within the insert is then increased to about 780 mm Hg, thickness doesn't increase to more than 1.1 cm but the width and the height can increase greater than 10% if the compliance of the material allows for this amount of increase. In some embodiments, the thickness of the material in these embodiments is less than 50 microns. In some embodiments, the thickness of the material in these embodiments is less than 25 microns. It has been discovered through the same experimentation which produced Table 2 that Inserts of these dimensions remain flat within the pericardium and produce minimal bulging (e.g. less than 1.0 cm) of the pericardium when inflated. They also take the curvature of the heart and do not collapse under their own weight. Furthermore, as discussed below, inserts with these dimensions and material thicknesses are implantable through access sheaths with diameters less than about 2.0 cm. In addition, as discussed below, inserts with these dimensions are removable from the pericardium after implantation as there is little ingrowth into the inserts with these thicknesses and dimensions.

In another embodiment, the compliance of the device is such that the pressure within the insert increases less than 5% with a 10% increase in volume. For example, when the dimensions of the insert are 10×15×1 cm (150 cm³) as above at 760 mm Hg (1 atm) and volume is introduced such that 165 ml are now within the insert, the pressure will not rise to be greater than 35 mm Hg within the insert. In another embodiment, the compliance of the device is such that the pressure rise within the insert is not greater than 5% with a 20% increase in volume. The volume increase in these examples is translated substantially to an increase in height and width and not an increase in thickness of the insert.

In any of these embodiments, the device can exhibit a curvature C (FIG. 10P) which is similar to the radius of curvature of a heart (2 cm to 10 cm). As described above, the curvature can result from plications 1550 made in the insert, from composite materials added to the insert, by creating pleats or connecting compartments between the walls of the insert, or from secondary structural materials placed within the inserts which have their own shape memory. These components and features of the insert allow the insert to maintain a substantially flat, curvilinear shape when fluid is introduced within the insert. Flat is not mutually exclusive from curvilinear. A shape can be flat in its thickness and have a curved plane. The flat shape has been found to be important in preventing remodeling of the pericardium over time.

In some embodiments, the elasticity, or the elongation of the material of the insert, can exhibit a strain of 200-300 percent or even up to 500 to 1000 percent. The elasticity determines the spring force with which the insert recoils as the heart begins its contraction phase. In one embodiment (FIG. 7C), the insert material is such that the insert can be elongated by greater than 200 percent and placed into an access sheath 995. The device is stretched like a rubber band and placed into an access sheath 970 so that the device in combination with the access sheath can be placed into the pericardium with a minimal incision in the skin of the patient. For example, the access sheath into which the device is placed may be less than one centimeter in diameter. The insert is then pushed through the access sheath and into the pericardial space. When the insert 980 enters the pericardium, the insert 980 contracts back down to its unstretched form 980. The unstretched form may not have fit through the access port unless stretched and expanded to >200% of its unstretched length.

In some embodiments, regions of the inserts can be made more rigid than other regions of the inserts. For example, lines or bars 2000 of a heavier material, can be placed on the inserts 2005, as shown in FIGS. 11D-11F so that when the inserts are expanded in the pericardial space, they expand in one direction and remain in place in this direction (e.g. in the longitudinal direction) along the heart wall from cranial to caudal. Such spines or rigid regions 2000 on the inserts allow the insert to attain a shape upon expansion which is pre-set to some extent by the spines. The rigid portions of the inserts or spines result in a degree of shape memory so that the inserts can grip the heart as the heart expands. The spines can be made from aplastic, a polymer, a metal, etc.

In one embodiment of an insert 2010, shown in FIGS. 11A-11C, a composite material is used in which a more rigid material such as polypropylene mesh 2020 or a polyester mesh is used, and a second, more biocompatible, flexible material such as a polyurethane is molded over the polypropylene.

FIG. 4 depicts an insert embodiment in which a composite skin is depicted. A first material 800 and a second material 700 is coated on first material 800. The second material can be a hydrophobic material such as PTFE or a lubricious material such as PVVF. In some embodiments, it may be desired to create a scarring effect between the implant and the outer surface of the heart. In this embodiment, material 700 is a mesh such as polypropylene which can induce ingrowth between material 800 and the epicardial surface of the heart. Either or both of the materials which make up the composite can be biodegradable. Exemplary biodegradable biomaterials include any of the biodegradable materials in patent applications nos, 20070071790, 20060264531, 20060193892, 20060188543, all of which are incorporated by reference into this application.

In another embodiment, the composite material has an insertable and/or removable component (FIG. 10I). For example, after the insert is placed into the pericardial space, a second component 1200 is placed inside the insert to increase the rigidity or create directionality 1230 of the insert inside the pericardium. This component 1260 can be a metallic component, a plastic component, a ceramic component, or a combination of these materials. The component 1200 can have shape memory so that for example, when the component is placed into the support structure, the shape memory component spreads the support structure in the pericardial space. After the insertion phase of the insert or component 1260 implantation, the purpose of the insertable component 1200 can be finished and the insertable component therefore removed 1250 from the insert: in other embodiments, the extra component of the insert is left in place to provide rigidity to the insert over time.

In another embodiment. FIG. 10J, the Insert has an outer shell 1300 and an inner component 1310 placed within the outer shell. These inner components 1310 can themselves be soft or rigid or fillable. The inner components can swell for example, if they are a hydrogel and they imbibe water from within the outer shell.

In addition to the polymers mentioned above, metals or metal alloys can be used in combination with polymers to support the heart wall. In some embodiments, the materials used need to have a space in which fluid can be placed to create a hydrostatic pressure within the insert. In one example, a fluid fillable insert is made from a polymer such as polyurethane, which in addition can have a nitinol mesh as part of the skin of the insert. In another embodiment, the insert has a stainless steel frame as part of the insert to aid in expansion and rigidity of the insert. Other useable metals include cobalt-chrome, stainless steel, and titanium.

In any of the embodiments, at least a portion of the insert can be biodegradable. For example, a coating or the skin of the insert or a part of the skin of the insert can be biodegradable. The biodegradable portion can be manufactured so as to degrade over a period of days, months, or years. In one embodiment, the insert is inflatable in addition to being biodegradable. Any of the biomaterials or biodegradable polymers mentioned in 20070071790, 20060264531, 20060193892, and 20060188543 (incorporated by reference into this application) can be used to create the insert.

In some embodiments, the insert comprises markers or regions for visualization from outside the patient. Such markers are visualizeable via one or more means such as fluoroscopy, MRL CT scan, and/or ultrasound echocardiography.

In some embodiments, a portion of the insert conducts electricity. The conductor can be a metal, ceramic, polymer, or a combination of these materials. The conductor can be used to run electrical current to the epicardium through the insert or around the insert to interact with the electrical conduction pathways of the myocardium. In one example, a current can be pushed through the material to defibriliate the heart to treat an arrhythmia. In another embodiment, electrical current is ran through the material to pace the heart. In another embodiment, electrical current is run through the insert to coordinate contractions of the left ventricle with the right ventricle or with one or more atria to synchronize or coordinate contractions of the heart. In another embodiment, electrical currents are gated to sensors which sense EKG signals. In this embodiment, subthreshold currents are run through the myocardium such as is discussed in (J. Cardiovascular Electrophysiology Vol. 15, pp. 418-427, April 2004 which is herein incorporated, by reference). In another embodiment, multiple electrical contact points to the epicardial surface are provided by conductors running through the insert. The multiple conductors can contact the epicardial surface in multiple points. An implanted controller or external controller can control which contact points are on or off.

In some embodiments, the insert is produced in part or in whole from a polymer or non-metallic material which conducts electricity. Current can then be run through the polymer to interact with the conduction pathways of the myocardium.

In another embodiment, electrodes are attached to a region of the heart; these electrodes are run along the pericardial insert white the pericardial insert remains free to float inside the pericardial sac.

In another embodiment, electrodes are independent from the structural portion of the insert. The electrodes can therefore have separate attachment points to the epicardium. In this embodiment, it may be desired to have the electrodes attach to the epicardium with a material which induces ingrowth into the electrodes. It may be desired that the separate or tethered structural portion of the insert then has a material with less ability to attach to the epicardium. For example, the insert may have a material such as PTFE, silicone, polyurethane, or a coating such that the structural portion of the insert minimally adheres to the epicardium but the electrodes adhere more tightly to the epicardium.

### Controlled Pericardial Effusion

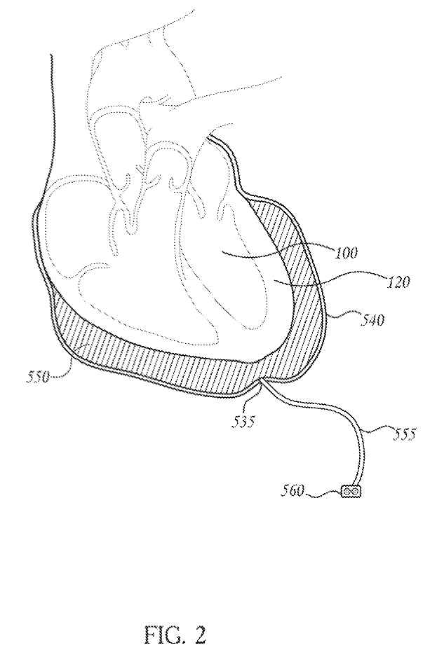

FIG. 2 depicts another embodiment of the current invention in which a fluid 550 is placed into the pericardial potential space without a balloon. The fluid can now freely move inside the pericardial space to exert a hydrostatic pressure on the myocardium. A port such as port 130 described above, may be used in this embodiment to communicate with the potential space so that fluid can be Injected and/or removed. Fluids such as saline may be utilized or thicker fluids such as silicone or mineral oil or hydrogels. In this embodiment, the pericardial space in combination with the port is a closed system in which fluid is held inside the pericardial space by the port which acts as a valve to selectively place or remove fluid from the pericardial space.

The port acts as a valve to control the volume and/or pressure inside the pericardial space. Similar types of sensors can be used as described above. For example, a pressure sensor inside the port can sense the hydrostatic pressure inside the pericardial space and based on this hydrostatic pressure, the amount fluid inside the space can be adjusted.

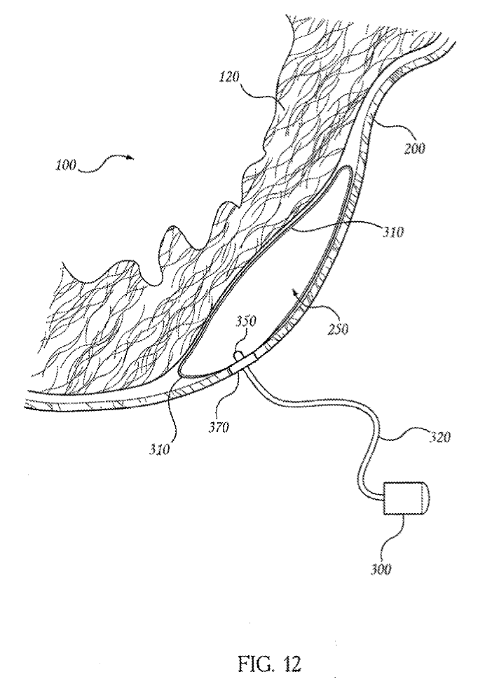

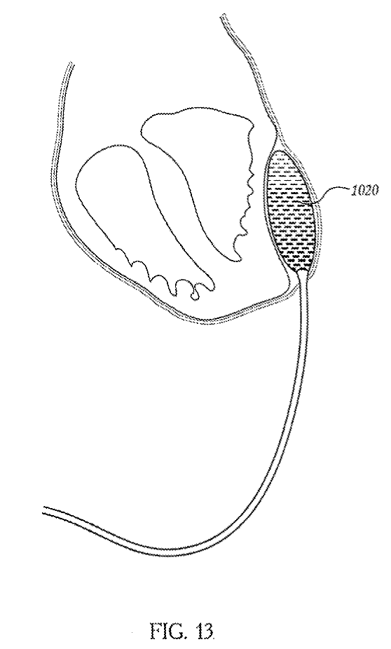

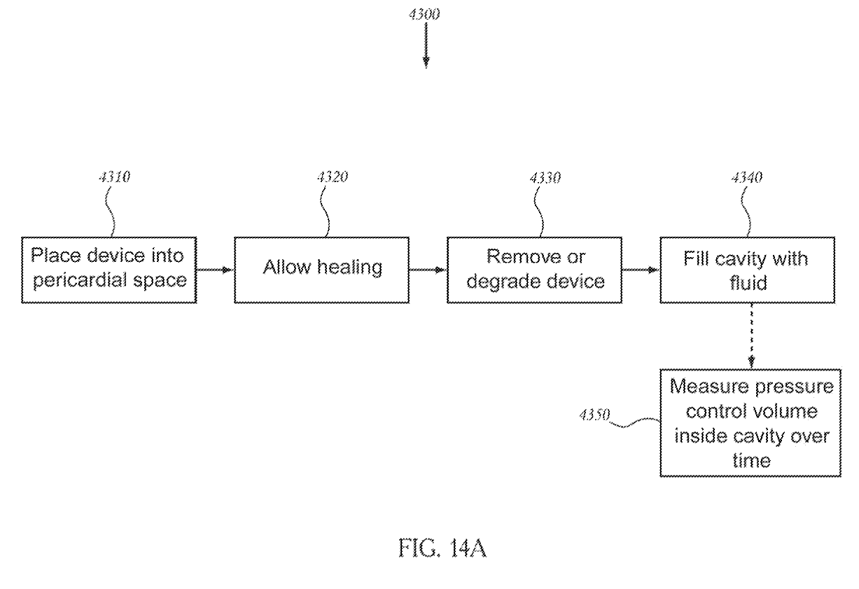

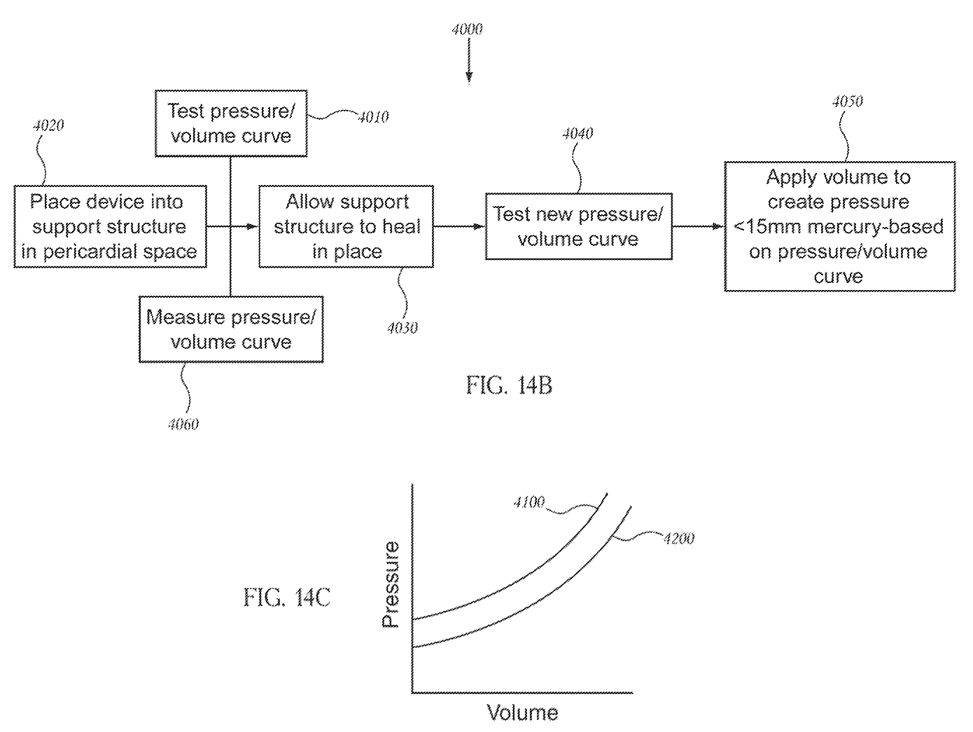

In another embodiment, (FIG. 12), a cavity 310 is created inside the pericardium which then retains fluid 250 and can also optionally attach to an inflation tube 320. This cavity 310 can be created in a lot of different ways. For example, a biodegradable material, can be placed into the pericardial space. As the biodegradable material degrades, a capsule forms around it. Once the biodegradable material is completely dissolved, capsule 250 is formed. A nozzle 350 communicates with the capsule 250. Rather than a foreign or prosthetic material interface with the myocardium, the capsule 310 now interfaces with the myocardium 120, residing between the pericardium 200 and the myocardium 120. In another example, the cavity is created with a temporary insert which is removed after a cavity is formed. In another embodiment, the cavity is created using staples, sutures, and/or other surgical implements.

Combinations with Electrical Modulation

FIG. 3 depicts another embodiment of the present invention in which electrodes 600 are placed on or near the inserts 150, 170. The electrodes work with the insert system to combine beneficial effects of constraint with those of resynchronization, pacing, defibrillation, or any other type of electrical modulation of cardiac tissue. The electrodes are also able to pace the heart or defibriliate the heart. The electrodes can also apply frequencies, currents, waves, and characteristic pulses which do not capture the electrical system of the heart but rather induce remodeling with a sub-threshold set of currents.

The electrodes can be placed on any region of the insert and can be held on the epicardium by the pressure of the insert itself in some embodiments. In other embodiments, electrodes are attached to the insert but are placed a distance from the insert on another region of the epicardial surface.

### Methods of Implantation

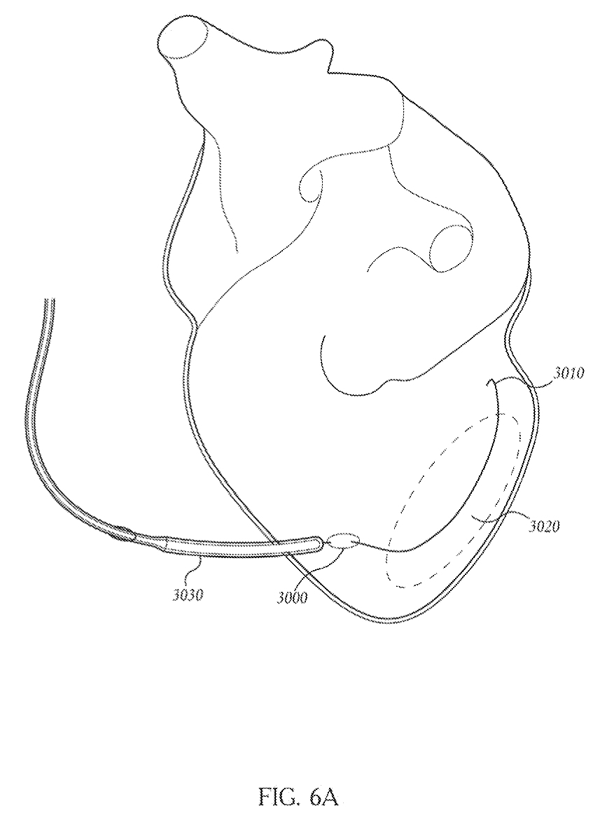

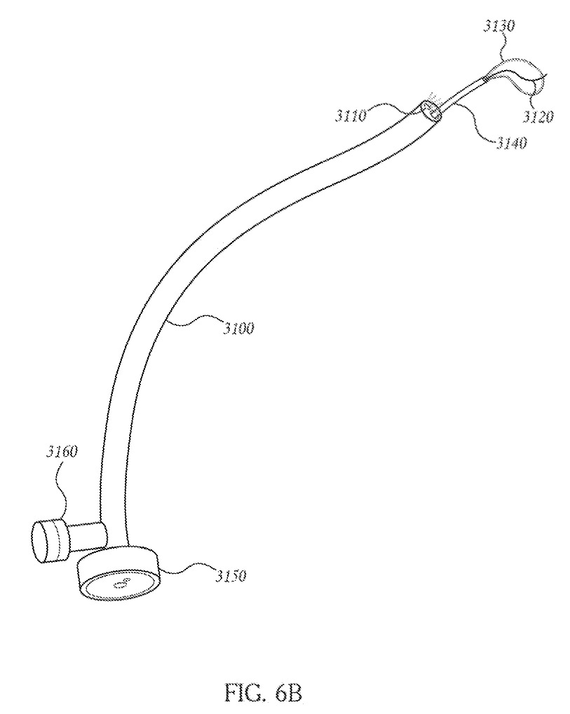

In one method to implant the pericardial inserts, an incision is placed in the skin of a patient and the sub-xyphoid region underneath the inferior sternum is accessed. From this position, the mediastinum can be entered to expose the pericardium. At this point in the procedure, a port can be placed through the skin incision and the port advanced to the pericardium. A camera may be used at this point in the procedure or a fluoroscopy machine can be used to visualize the direction of the port relative to the target region on the epicardium. A small hole (FIG. 6; 3000) may then be made in the pericardium and a camera (FIG. 6B; 3110) placed within the pericardial space to visualize placement of the insert. In the case where fluoroscopy is used, a mobile fluoroscopy machine may be utilized to determine the direction of the port and a fluroscopically visible marker may be placed at the end of the port. The camera may be a CCD camera, a CMOS camera or a fiber optic endoscope 3100. The camera 3110 may be placed at the end of a flexible tube or at the end of a rigid tube. In some embodiments, an endoscope or camera is used when adhesions have formed inside the pericardium or a patient has undergone a bypass operation in the past. Adhesions can be visualized and/or lysed with a balloon or electrosurgical device place through an endoscope and inside the pericardial space.

In another embodiment, the insert is placed percutaneously. In this embodiment, a needle with a lumen is placed inside the skin and then a guidewire (FIG. 6A; 3010) is threaded through the needle and into the pericardial space. An access catheter 3030 can then be placed inside the pericardium between the epicardial surface of the heart and the inner portion of the pericardium. The insert is then threaded over the guidewire and into the space between the epicardial surface and the surface of the pericardium. The diameter of the access catheter can be from 5F to 35F or from 1 mm to 15 mm in diameter. The thickness of the insert upon insertion through the access catheter is smaller than when the insert leaves the access catheter. The guidewire can range in thickness from 0.010 inches to 0.100 inches.

The access catheter 3030 can be flexible in some embodiment such that it can negotiate bends without kinks. The access catheter in some embodiments can act like an endoscope where the direction of the access catheter is controllable by the operator on the proximal end of the endoscope. In some embodiments, the access catheter is an endoscope 3100 (FIG. 6b). Such an endoscope can be supplied with different diameters. For example, endoscopes can be supplied with diameters from 3 mm to 5 mm or from 2 mm to 9 mm or from 5 mm to 15 mm. In this embodiment, the endoscope can be placed over a guidewire 3120 in some embodiments, under direct visualization with a camera 3110; in another embodiment, the endoscope is placed directly into the pericardial space without the use of guidewire 3120. Access sheath 3140 can be utilized to deploy insert 3130. Control knob 3160 can be used to direct the distal portion of the endoscope and camera interface 3150 can be utilized for direct visualization or for attachment to a camera. In these embodiments, the endoscope can be utilized to directly visualize the region on the myocardium where the device is to be placed and to lyse adhesions which may have formed inside the pericardium.

The insert can be less than about 1.5 cm in its compressed state or less than 0.75 cm or even less than 0.5 cm in its compressed state inside the access catheter or endoscope. In the expanded state inside the pericardium, the surface area of one face of the insert can be greater than 5 cm² or greater than about 10 cm². In some embodiments where the heart is enlarged and in heart failure, the surface area of the insert can be greater than about 20 cm². The thickness of the insert material can range from 10 microns to 20 microns or from 5 microns to 20 microns or from 10 microns to 40 microns or from 30 microns to 100 microns. Creating an insert with thicknesses in this range allow the device to be placed into a small diameter access catheter or endoscope for delivery into the pericardial space; in addition, such thin structures are required to fit into the thin potential space between the pericardium and the epicardium while still allowing for fluid to be placed inside the inserts. As described below, a polyurethane blend is used in one preferred embodiment because it provides great strength at a low material thickness. Inserts with wall thicknesses less than 25 microns have been manufactured and can slide easily into the pericardial space.

In some embodiments, the insert is placed into an access sheath so that it deploys in a substantially planar or two-dimensional manner. For example, in FIG. 7B, insert 3070 is shown deploying from an access catheter 3080 in a substantially two dimensional manner. As opposed to unrolling, the insert 3070 is folded like an accordion and then deployed like an accordion or a fan would expand. Although the implant can fit the curvature 3090 of the heart, the insert can deploy from the access catheter in two directions as opposed to unrolling from the catheter. The insert 3070 in this embodiment is structured with pleats so that it unrolls in this manner.

After the camera is placed inside the pericardium and/or fluoroscopy is begun, a guidewire 3010, 3120 may be placed inside the pericardium and positioned over the region of the myocardium to be treated.

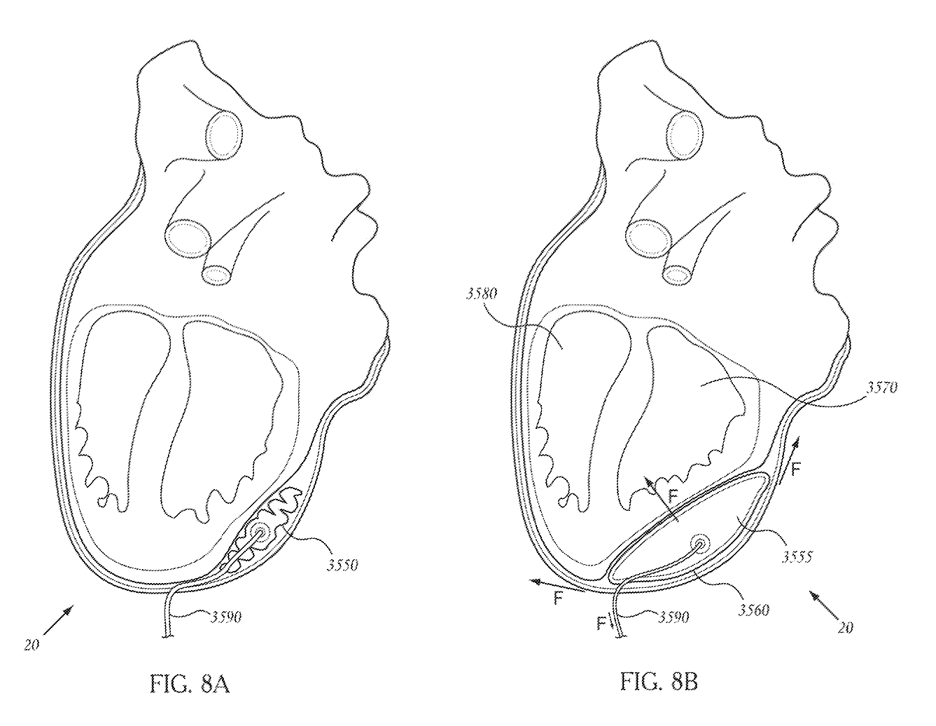

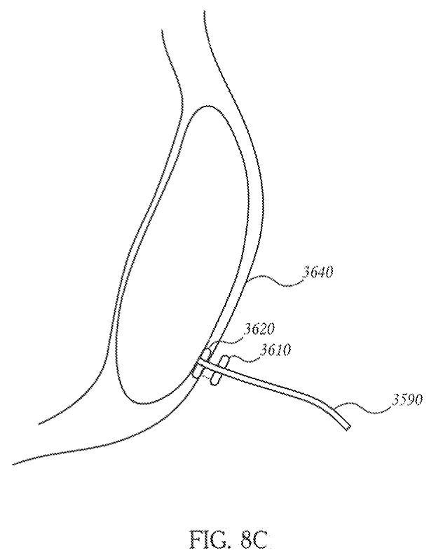

The insert 3050 may then be advanced over the guidewire 3010 and placed inside the pericardium 3030 between the epicardium 3020 and the pericardium 3030 (FIG. 7A). As described above, the insert can be secured to the pericardium 3030 or the epicardium; in another embodiment, the insert can be left to “float freely” between the pericardium and the epicardium. Of course, the insert 3050 will not float but will be held against the epicardium 3020 by forces F shown in FIG. 8B. Over time, the insert will heal into place and stabilize within the pericardial space. In an embodiment where the insert is two-piece and self-assembles (e.g. by magnets), the second portion of the insert is placed into the pericardium after the first portion. With the two components of the insert in the pericardial space, the magnets allow them to forcibly connect with one another. In some embodiments, two independent inserts are placed inside the pericardium, one after the other, as shown in FIG. 1C. In this embodiment, the independent Inserts each heal within the pericardium and essentially create a structure surrounding the heart but without being continuous. Each insert can then apply independent forces to the left and right ventricles respectively but without direct fluid communication between the two.

FIG. 8A depicts a cross-sectional view of the heart 20 with insert 3550 in the undeployed configuration. FIG. 8B depicts the insert in its deployed state 3560. The deployment occurs by filling the insert 3560 with fluid as described above. In some embodiments, the insert is deployed by pulling back a sheath over the insert, then subsequently filling the insert with fluid 3550. As shown in FIG. 8B, when the insert is fully expanded, the pericardium applies force F to the insert and subsequently to the left ventricular chamber 3570. As described below and revealed in Table 1, insert 3560 can apply a force to the left ventricular chamber 3570 and the right ventricular chamber 3580 will not see the same force F. A differential pressure can therefore be applied to the left ventricular chamber than to the right ventricular chamber. In addition, force can be shunted or transferred along the stem 3590 from the implant to direct force in a direction away from the heart.

Subsequent to placement of the insert, the tie line or access port to the insert may be run through the hole in the pericardium (pericardotomy) and connected to a subcutaneous access port 1300 (FIG. 9A). The access port 1300 allows for fluid administration or removal from the insert. A separate system or structure 1310 is optionally included and in some embodiments is integral to the port. This system or structure 1310 can be used for sensing or application of electrical current to the heart for pacing, defibrillation, rhythm monitoring, etc.

In some embodiments, the insert floats freely but one or more fluid line(s) (160, 180 in FIG. 1A) are attached to the pericardium. The fluid lines can be rigid or have a rigid component so that the attachment to the pericardium allows maintenance of the position of the pericardial insert inside the pericardial space. In another embodiment, a washer like device 3610 is positionable over the fluid line 3590 to grip the pericardium and/or fluid line from outside the pericardium, as shown in FIG. 8C. This configuration will assist in holding the insert in place within the pericardial space. In another embodiment, an expandable component is placed over the fluid line and into the pericardial space or is placed over the fluid line in a configuration with a small profile, then expanding to a larger profile 3620 after placement inside the pericardial space. After expansion inside the pericardial space, the expandable component is pulled against the pericardial membrane 3640 and an outside component is placed over the fluid line to mate with the inner expandable component with the pericardial membrane in between; in this manner, the insert is locked in place within the pericardial space.

In another embodiment (FIG. 15A), electrodes are held in place by the insert 5010. In this embodiment, electrodes are placed inside the pericardium of a heart and a support structure, insert, inflatable insert, etc. 5010 is placed next to the electrodes 5000 to hold the electrodes in place. In some embodiments, it may be desirable for the electrodes 5000 and insert 5010 to have different healing mechanisms (based on materials or chemicals placed on or near the electrodes) so that one of the structures heals inside the pericardium separately from the other. For example, in FIG. 15A, insert 5010 is shown pushing electrodes 5000 against the surface of the heart 5005. The electrode material can be ones which promote ingrowth such as titanium, stainless steel, or nithiol mesh. The materials can also be conducting polymers or polymer-metallic composites such as polypropylene which will facilitate the healing process. FIG. 15B depicts the insert after the healing process has occurred. The electrodes 5000 are now healed into the epicardium or myocardium and the insert 5010 remains separable from the electrodes 5005. If the electrodes 5000 and the Insert 5010 healed together, the desirable properties of each may not be attained. The desirable property of the insert is removability and adjustability and of the electrodes is that they be able to conduct current to the heart. It may be desirable for the electrodes to be fixed to the heart so that sensory function or conduction can be achieved with small currents. On the other hand, it may be desirable that the insert or support structure be removable after a specific period of time. In this case, it may be desirable that the insert portion be able to compress the electrode against the epicardium and that the electrode heal into the heart but that the pericardial insert be removable from the heart. In this embodiment, the materials of each would be distinct and customized for the application of each. The electrodes 5000 may have certain features which allow for implantation into the epicardium 5005. For example, the electrodes may have an attached mesh which leads to fixation attachment of the electrodes against the epicardium 5005. In another example, the electrodes have a screw type mechanism in which the electrodes can be twisted into the myocardium. Once the electrode and support structure are in place, the electrode can heal separately into the epicardium while the support structure heals within the pericardium. Each therapeutic mechanism can be used separately or in combination. It is important however that each therapeutic mechanism be allowed to heal in place independently of one another.

A method of electrode placement is depicted in FIG. 15C. The device or insert is expanded 5050 in the pericardial space to place the electrode against the heart 5005. Healing of the electrode against the heart is achieved typically in 7-10 days 5060. At this time, the insert can be partially or fully deflated 5070 to separate from the electrodes.

Other Methods to Treat Heart Failure with Pericardial Inserts

In other embodiments, the access port to the insert is utilized for other treatments. Because the port is accessible over time, the support structure and therefore the myocardial wall can be accessed over time as well through the port. The port can comprise a power supply or a sensor and can further comprise intelligence through a microprocessor.

In other embodiments, the inserts are used to apply other types of energy to the myocardium. For example, radiofrequency energy is applied subcutaneously and through the insert to affect the myocardium.

In another embodiment, light energy is applied to the myocardium through the insert or through the fluid of the insert. For example, a fiber optic can be placed through the support and into the insert to apply light therapy to the myocardium. Light therapy can include visible, ultraviolet, and/or infrared light therapy or combinations thereof. The light can activate or deactivate materials associated with the support structure. For example, light can be used to cross-link and reinforce a capsule created by the insert or the light can be used to cross-link a polymer which is placed into the capsule created by the insert. The insert should be transparent to these wavelengths of light in order to be useful. In one embodiment, infrared light is applied through the insert to aid in healing of the myocardium.

In another embodiment, heat energy is applied through the inserts to treat the myocardium.

In another embodiment, heat energy is removed from the insert to cool the myocardium.

In another embodiment, electromagnetic energy is applied to the pericardium through the insert.

In another embodiment, pharmaceuticals or other bioactive materials are released from the insert. In this embodiment, an extra lumen can be placed on the insert or associated with the insert. This lumen can communicate with the area outside the insert so that when the pharmaceuticals are pushed through the lumen, they are localized in the vicinity of the insert. Pharmaceuticals and bioactive materials include cardiac altering drugs as well as structural materials or other materials which can affect the heart, such as stem cells.

In another embodiment, a drug releasing patch or healing patch is placed on the inner surface of the insert to aid in healing the heart.

In another embodiment, stem cell therapy is applied to the heart using the insert.

### Treatment of Injured Myocardium

In another embodiment, injured myocardium is treated with or without treating dilatation of the heart. For example, an insert can be placed on or near injured myocardium and the myocardium supported by the insert. The insert can be placed at the time of an infarct or some time after an infarct. The support or the medicine release by or in vicinity of the insert is therapeutic for the infracted region. Treatment of an infarct region prior to dilatation is preferable in some embodiments as opposed to treatment of the heart after remodeling has occurred.

### Treatment of Valvular Disease

In another embodiment, treatment of valvular disease is described. For example, an insert is placed at least partially around the mitral valve annulus to alter the shape of the mitral valve over time.

### Experimental Verification

To verify the physiologic principles above, a series of experiments was performed. A flexible and expandable polyurethane balloon with a wall thickness less than 50 microns was inserted into the pericardium in a porcine animal model. In addition, the balloon had a polyurethane tubing which was thermoset to the curvature of the heart. The tubing was semi-rigid to assist in holding the insert in place as well as prevent the insert from collapsing under its own weight. A pressure measuring catheter was inserted into both the left and right ventricles. The motion of the heart walls was followed with echocardiography. The balloon was inserted over the region of the left ventricle and sequentially filled with 10 cc, 20 cc, 30 cc, 40 cc, 50 cc saline . . . up to 160 cc. When the device contained less than 100 cc of volume at atmospheric pressure, the thickness of the device was less than 1.0 cm. The dimensions of the device were less than 10×10 cm². The radius of curvature of the device was from 2 cm to 10 cm depending on the number of pleats connecting the two surfaces of the device together and the degree of curvature of the thermoset polyurethane spine. The pericardium at these filling volumes was stretched and the balloon was compressed against the left ventricle with a pressure defined by the volume within the insert. As saline was introduced into the expandable balloon, the left ventricle became progressively compressed so that it is prevented from completely filling. At the same time, the right ventricle continued to fill normally. See Table 1 below for detailed data. Pressure data in Table 1 is expressed as systole/diastole (mean over time), with all pressures provided in mm Hg.

TABLE 1

| | Volume | | | | | | | | | | |
|---|---|---|---|---|---|---|---|---|---|---|---|
| | | | | | | | | | | | balloon |
| | baseline | 25 cc | 35 cc | 45 cc | 60 cc | 80 cc | 90 cc | 120 cc | 140 cc | 160 cc | out |
| Left | 80/7 (40) | 70/13 (35) | | 75/12 (37) | 78/13 (41) | 69/13 (35) | 75/20 (43) | 68/20 (44) | 62/11 (36) | 57/14 (34) | 80/15 (45) |
| Ventricle | | | | | | | | | | | |
| Pressure | | | | | | | | | | | |
| Right | 25/8 (15) | 27/10 (18) | | 27/11 (17) | 27/12 (19) | | 25/10 (17) | 26/10 (28) | 26/12 (18) | 26/14 (20) | 26/12 (20) |
| Ventricular | | | | | | | | | | | |
| Pressure | | | | | | | | | | | |
| Balloon | NA | 12/8 (10) | 15/10 (13) | | 15/10 (12) | 22/15 (18) | 35/20 (25) | 25/15 (18) | 40/25 (28) | 26 | 0 |
| Pressure | | | | | | | | | | | |
| Cardiac | | 4.4 | | | 6.2 | | 5.9 | 4.6 | 4.5 | 4.7 | 8 |
| Output | | | | | | | | | | | |
| Heart Rate | 90 | 60 | | 63 | 65 | | 67 | 71 | 73 | 80 | 70 |
| Echo EF | 55% | 50% | | | 55% | | 55% | | | 35% | 55% |
| SVO2 | 72% | 55 | | | 63% | | 65% | 63% | 65% | 57% | 79% |
| Wedge | 13 | 17 | | | 19/15 (16) | | 20/10 (15) | 25/17 (19) | | 21/18 (20) | 15/13 (14) |
| Pulmonary | 20/10 | | | | | | | | | | |
| artery | | | | | | | | | | | |
| pressure | | | | | | | | | | | |
| Comments | | | | | | | | | | Low voltage | EKG |
| | | | | | | | | | | EKG | normalized |
| | | | | | | | | | | reading | |

After the 160 cc volume caused almost complete collapse of the left ventricle, the balloon was removed from the pericardium. The left ventricle immediately returned to its pre-balloon form in which the left ventricle vigorously contracted. As can be seen in Table 1, the ejection fraction (measure of the functioning of the heart) increased to its pre-balloon levels. Similarly, the mixed venous oxygen saturation (measure of cardiac output) returned to its pre-balloon levels. The pressure inside the ventricle decreased as the balloon was filled and similarly returned to its baseline state after the balloon was removed. The maximum pressure inside the pericardial balloon was 28 mm Hg which was high enough to cause the hemodynamic compromise (decreased systemic blood pressure) seen in Table 1. Therefore, in this experiment, the useful range of pressure inside the insert is below 28 mm Hg and above zero. More specifically, a useful range of pressure is below 18 mm Hg and above 5 mm Hg at end-diastole.

Follow up after 60 days with the support structure in place indicates that the support can continue to modulate the wall stress of the ventricle on a chronic basis (Table 2). These chronic data show that regions of the wall of the heart can be selectively treated while not treating other regions of the heart and that this ability continues over time after the implant.

TABLE 2

| Insert | | | | |
|---|---|---|---|---|
| Volume | Insert Pressure | LV pressure | RV pressure | LV diameter |
| baseline | 12/8 (10) | 90/10 | 30/13 | 3.2 |
| Add 5 cc | 28/18 (24) | 80/28 | 28/12 | 2.5 |
| Subtract 5 cc | 5/0 | 90/8 | 28/10 | 3.4 |
| Subtract 2 cc | 10/5 | 85/12 | 28/10 | 2.8 |

Table 2: The effect of time on the ability of the insert to modulate pressure over the epicardium and ultimately the myocardium. These data are from an insert after it was implanted for 8 weeks. The pressure within the insert increases over time despite a constant volume within the insert as depicted in the “baseline” above. At the initial implantation, the pressure within the pericardium was left at 10/5 with a mean of 6. The pressure inside the insert correlates to the left ventricular pressure which increases proportionately. It can be seen in this table that the pressure rose over time (12/8 vs 10/5) inside the insert vs. the pressure at the time of the implantation. In addition, a small added volume significantly increased the left ventricular end diastolic pressure; the compliance of the insert has effectively been decreased because of a capsule formed around the insert. The increase in opening pressure inside the insert and the increase in the pressure-volume curve was due to the formation of the capsule around the insert. The capsule is caused by a healing process around the insert. The capsule in combination with the insert is what ultimately modulates the force on the epicardium and is modulated by the fluid inside the insert; therefore the material properties of this combination change with time. Curve 4200 in FIG. 14C depicts the pressure-volume relationship at implantation and curve 4100 depicts the pressure-volume relationship after healing occurs.

A clinically useful method of use based on these data would be to insert the device into the pericardium with minimal fluid; after healing occurs and the compliance curve of the system has shifted, increasing amounts of fluid would be introduced into the system to create the desired effect on the heart. At each adjustment time point, the pressure-volume (compliance) curve would be determined and the appropriate pressure at the time point applied to the epicardium.

Further follow up reveals that the initial pressure created in the support structure holds the support structure in place while the support structure heals into place within the pericardium.

Follow up at sixty days reveals that a polyurethane material fashioned into an inflatable structure with wall thicknesses less than 50 microns and which is placed inside tire pericardial space indeed maintains its shape over time and is indeed removable. The thin balloon further did not create significant scarring of the pericardium, the pericardium retaining its see-through qualities and not showing great amounts of inflammation, either acute or chronic. The thin polyurethane balloon did however induce enough granulation tissue to maintain its position inside the pericardium. The implant spine prevents the insert from migrating and/or from collapsing under its own weight. The thin balloon (for example, less than 1.0 cm thick and with wall thickness less than 50 microns when less than 100 cc is placed at atmospheric pressure), served the purpose of fitting inside the pericardial space through an access sheath less than 2 cm and providing force on the heart as the heart expands during diastole.

Introduction of Fluid Directly into the Potential Pericardial Space

FIG. 2a depicts a section of a heart. Cardiac chamber 100 is the inner region of the heart where blood enters and then is pumped out. The pericardial potential space can be filled with fluid 550 and is wholly contained in the sense that it can be filled with fluid under pressure. The outer region of the pericardial space is the pericardium 540. A fluid delivery catheter 555 allows for communication between a port 560 and the nozzle 535. The nozzle allows fluid to be pushed into the pericardial space 550. Port 560 is designed to be placed inside the patient or outside the patient. It can be implanted in the subcutaneous region or in the abdominal or chest cavity. Fluid can be injected through the port from outside the patient to the pericardial potential space 550. The fluid can be placed under a known and controllable pressure to control expansion of the myocardium 120 and prevent the unstable situation during heart failure.

A pressure monitoring system can be used in this embodiment. Alternatively, the pressure is monitored through the port with a pressure monitor. If the fluid inside the pericardium is greater than about 5-15 mmHg, the patient and the patient's doctor/s can be warned.

Introduction of Fluid into an Encapsulated Region Inside the Pericardial Space

In another embodiment (FIG. 12), a flexible cavity 250 is created inside the pericardial space. The cavity 250 is created after introduction of an insert into the pericardial space wherein the insert is then covered with granulation tissue 310. It has been discovered that an inflatable material, a solid material, a porous material, a biodegradable material, a drug eluting material, or a coated material can form a natural capsule within the pericardium. This capsule can communicate with a permanent or semi-permanent insertion tube and be inflated or deflated over time and in response to certain pressure, volume, or other physiologic changes within the heart.

In this embodiment, the insert can be removed after such time that the granulation tissue 310 has formed on, around, or through the insert. In this embodiment, it is possible that after the insert portion is removed or that the material of the insert dissolves, a communication with the granulation tissue/cavity 310 is maintained with a fluid nozzle 350 and connector 370 which connects the fluid nozzle 350 to the pericardium 200. In this embodiment, the granulation cavity tissue cavity 310 can be pressurized, inflated, or deflated over time. In some embodiments, the insert is left in place after the granulation tissue 310 has formed on or around the insert and the insert is used to increase pressure or tension on the granulation tissue 310.

What is claimed is:

1. A method of remodeling a heart comprising:
forming a tissue capsule within an intrapericardial space of the heart; and
adjusting the pressure exerted by the the capsule on the heart.

2. The method of claim 1 wherein the forming step comprises placing a material into the intrapericardial space.

3. The method of claim 2 wherein the material comprises an expandable device.

4. The method of claim 3 wherein the expandable device comprises an inflatable device.

5. The method of claim 4 wherein the forming step comprises inflating the inflatable device.

6. The method of claim 5 wherein the adjusting step comprises inflating or deflating the device.

7. The method of claim 2 wherein the placing step comprises delivering the material to the pericardial space through a delivery sheath having diameter of 2 cm or less.

8. The method of claim 7 wherein the placing step further comprises moving the material from a delivery configuration within the sheath to a deployed configuration outside of the sheath and within the intrapericardial space, the material in its deployed configuration having a greater length in at least one dimension than in its delivery configuration.

9. The method of claim 2 further comprising removing the material after forming the capsule.

10. The method of claim 2 wherein the placing step further comprises visualizing placement of the material with a camera inside the intrapericardial space.

11. The method of claim 2 wherein the placing step further comprises visualizing adhesions with a camera inside the intrapericardial space and further comprises cutting said adhesions from within the intrapericardial space.

12. The method of claim 1 wherein the adjusting step comprises inflating or deflating the capsule.

13. The method of claim 1 further comprising reinforcing the capsule with a high viscosity or high molecular weight material.

14. A method of remodeling a heart comprising:
inserting a delivery sheath into an intrapericardial space between a pericardium and a myocardium of the heart, the delivery sheath having a diameter of 2 cm or less;
moving a pericardial device from a delivery configuration within the sheath to a deployed configuration within the intrapericardial space, the intrapericardial device in its deployed configuration having a greater length in at least one dimension than in its delivery configuration; and
controlling the pressure from the pericardium onto the intrapericardial device and from the intrapericardial device onto the myocardium.

15. The method of claim 14 further comprising permitting the intrapericardial device to move from the delivery configuration at least partially to the deployed configuration without outside actuation.

16. The method of claim 14 further comprising expanding the intrapericardial device from the delivery configuration to the deployed configuration.

17. The method of claim 16 wherein the expanding step comprises inflating the intrapericardial device.

18. The method of claim 14 further comprising loading the intrapericardial device into the delivery configuration within the sheath, the loading step comprising lengthening at least 200% in another dimension from an at-rest configuration outside of the sheath.

19. The method of claim 14 further comprising forming a tissue capsule around at least part of the intrapericardial device.

20. The method of claim 19 further comprising adjusting the size of the capsule.
